(19) United States
(12) Patent Application Publication
(10) Pub. No.: US 2020/0087394 A1
(43) Pub. Date: Mar. 19, 2020

(54) ANTIBODIES TARGETING A LIGAND FROM AN IMMUNE CHECKPOINT, WITH AN FC FRAGMENT HAVING AN IMPROVED AFFINITY FOR CD16A

(71) Applicant: LABORATOIRE FRANÇAIS DU FRACTIONNEMENT ET DES BIOTECHNOLOGIES, LES ULIS (FR)

(72) Inventor: Céline MONNET, LAMBERSART (FR)

(21) Appl. No.: 16/495,427

(22) PCT Filed: Mar. 19, 2018
(86) PCT No.: PCT/EP2018/056891

§ 371 Date: Sep. 19, 2019

(30) Foreign Application Priority Data
Mar. 20, 2017 (FR) 1752285

(51) Int. Cl. C07K 16/28; A61P 35/00

(52) U.S. Cl. CPC C07K 16/283; C07K 16/2818; C07K 16/2827; C07K 2317/732; C07K 2317/41; C07K 2317/52; C07K 2317/72; A61P 35/00

(57) ABSTRACT

Disclosed is an antibody targeting at least one ligand from an immune checkpoint, having a region Fc that is mutated in relation to that of a parent antibody and has an improved affinity for the receptor FcgRIIIa (CD16a) and/or a higher ADCC activity than the parent antibody.

The present invention concerns cancer immunotherapy.

### TECHNOLOGICAL BACKGROUND OF THE INVENTION

Immunotherapy, which entails administering exogenous antibodies to patients, is currently widely used to treat various pathologies and cancers in particular.

In recent years, knowledge of the biology and immunology of cancers has constantly progressed. It is henceforth recognised that the immune system is involved in antitumor response, particularly through recognition of cancer cells by the immune cells. This recognition can lead to the controlling and even elimination of tumours. However, immune effector cells have receptors on their surface known as immune checkpoints. The purpose of these receptors is to modulate (inhibit or activate) immune response and in particular to maintain self-tolerance. It is now known that cancer cells borrow this escape mechanism to resist immune response, in particular through expression on their surface of ligands of the receptors of said immune checkpoints, which will lead to inhibition of the response of the immune effector cell when it recognises the latter (Pardoll et al., Nat. Rev. Cancer. 12:252-264 (2012)). A new approach to cancer immunotherapy has therefore emerged to counter-attack this phenomenon of immune escape by restoring immune response and hence tumour rejection. In the last few years, antibodies directed against these immune checkpoints have been developed. The chief antibodies that are marketed or under development target:
Cytotoxic T-Lymphocyte Associated antigen 4 (CTLA4): this is the case for Ipilimumab (Yervoy®, Bristol Myers Squibb). This monoclonal antibody causes blockading of the CTLA4 receptor, an inhibitory immune checkpoint present on T lymphocytes, and consequently leads to activation of the immune response of said T lymphocyte. In other words, Ipilimumab blockades the immune escape route of cancer cells by suppressing their inhibitory action on T lymphocytes via their stimulation of the CTLA4 receptor. A clinical study allowed the evidencing, for the first time, that patients suffering from metastatic melanomas treated with an anti-CTLA4 antibody (Ipilimumab), have their lifetime increased (Hodi et al., N Engl. J. Med., 363: 711-723 (2010) and 363:1290 (2010) (erratum); Robert et al., N Engl. J. Med., 364: 2517-2526 (2011));
Programmed Cell Death Protein 1 (PD1): this is the case for Nivolumab (Opdivo®, Bristol Myers Squibb) and Pembrolizumab (Keytruda®, Merck). This monoclonal antibody causes blockading of the PD1 receptor, an inhibitory immune checkpoint present on T lymphocytes, leading to activation of the immune response of said T lymphocyte. In other words, Nivolumab blockades the immune escape route of cancer cells by suppressing their inhibitory action on T lymphocytes T via their stimulation of the PD1 receptor. In particular, Nivolumab appears to have antitumor activity in patients suffering from metastatic renal cell carcinoma (Motzer et al., American Society of Clinical Oncology 33 (13): 1430-1437 (2014)); or
Lymphocyte Activation Gene 3 (LAG3) (e.g. BMS-986016). LAG3 is an inhibitory immune checkpoint present on T lymphocytes T. It is currently the target of inhibitor development and in particular of monoclonal antibodies. Similar to CTLA4 and PD1, the blockading of this receptor would allow the blockading of its inhibiting effect on the response of effector cells, thereby activating immune response.

More recently, antibodies directed against ligands of immune checkpoints have been developed. The chief antibodies marketed or under development target the ligand of PD1 (Programmed Cell Death Protein 1) contained on antigen presenting cells, and tumour cells. For example, Atezolizumab/Tecentriq (Genentech/Roche) is an anti-PDL1 antibody and was granted approval in May 2016 for the treatment of patients with bladder cancer. This antibody is an IgG1 modified to be aglycosylated, without effector activity. Nevertheless, to date, most patients show little or no response to such treatment via anti-checkpoint ligand antibodies. In addition, some patients face toxicity reactions in the body after receiving treatment. For example, it was observed in some patients that anti-PDL1 antibodies induce autoantibodies in patients, related to the onset of skin and liver problems for example. Finally, it was observed that some patients become resistant to the treatment.

At the present time there is a need to optimise the immunotherapy approaches used to blockade these immune checkpoints, notably with a view to obtaining a more efficient and/or less toxic clinical response. There is a particular need to obtain antibodies directed against an immune checkpoint ligand, having improved effector properties and advantageously having an improved half-life in the body allowing extended antitumor effect, whilst being well tolerated by the body. In particular, said antibodies with improved half-life can advantageously be administered in lower dosage with the same or greater efficacy, which would allow limiting of the side effects observed in some patients.

### SUMMARY OF THE INVENTION

The present invention therefore relates to an antibody directed against at least one ligand of an immune checkpoint, having a modified Fc region compared with that of a parent antibody, having improved affinity for the FcgRIIIa (CD16a) receptor and/or increased ADCC activity compared with a parent antibody. Said antibody is also called an «anti-ligand antibody». It is adapted for use in cancer treatment.

The invention also relates to an anti-ligand antibody composition, and to a pharmaceutical composition comprising at least one antibody of the invention (anti-immune checkpoint or anti-ligand). Said composition can be suitable for use in the treatment of cancers.

The present invention also relates to products containing:

a) an antibody directed against a ligand of an immune checkpoint, or a composition of antibodies directed against a ligand of an immune checkpoint, and

b) an antibody directed against an immune checkpoint, having a modified Fc fragment compared with that of a parent antibody, having improved affinity for the FcRn receptor and optionally reduced functional activity mediated by the Fc region, said immune checkpoint being selected from among PD1, CTLA4, TIM3, LAG3, KIR, BTLA1 and a2AR, as combination products for simultaneous, separate or time-staggered administration, for use thereof in the prevention or treatment of cancers.
The invention also concerns:
   an anti-immune checkpoint antibody for use thereof in the prevention or treatment of cancers, in combination with an anti-ligand antibody or anti-ligand antibody composition; and
   an anti-ligand antibody or anti-ligand antibody composition for use thereof in the prevention or treatment of cancers in combination with an anti-immune checkpoint antibody.

### KEY TO FIGURES

FIG. 1 gives the alignments of native human IgG1 sequences referring to positions 216 to 447 (as per EU index) with the corresponding human IgG2 (SEQ ID NO: 7), human IgG3 (SEQ ID NO: 8) and human IgG4 (SEQ ID NO: 9) sequences. The IgG1 sequences refer to the G1m1,17 allotype (SEQ ID NO: 6) and G1m3 allotype (SEQ ID NO: 10). The “CH2-CH3 lower hinge” domain of IgG1 starts at cysteine 226 (see arrow). The CH2 domain is highlighted in grey and the CH3 domain is in italics.

FIG. 2 shows the glycan structure of the G0, G0F, G1 and G1F forms.

FIG. 3 shows the expression vectors containing the heavy chain (HC) of anti-PDL1 mutated on the Fc fragment, and the non-modified light chain (LC) of the anti-PDL1 antibody under consideration. FIG. 3A illustrates the IGG1AV-WT and IGG1D-WT vectors, whilst FIG. 3B illustrates the IGG1A-WT and pCEP4 vectors.

«IGG1AV-WT» corresponds to the expression vector encoding avelumab; «IGG1D-WT» corresponds to the expression vector encoding durvalumab; and «IGG1A-WT» corresponds to the expression vector encoding atezolizumab.

### DETAILED DESCRIPTION

### Characteristic of the Antibodies

The present invention relates to an antibody directed against a ligand of an immune checkpoint (anti-ligand antibody), having a modified Fc region compared with that of a parent antibody, having improved affinity for the FcgRIIIa receptor (CD16a) and/or increased ADCC activity compared with the parent antibody.

The anti-immune checkpoint ligand antibody allows binding with the target tumour cell. For example, by binding to the ligand present on tumour cells (e.g. PD-L1), it allows the recruiting of effector immune cells via the mutated Fc region (having an effector function). This leads to direct cytotoxicity on the tumour cells.

The present invention preferably relates to an antibody directed against a ligand of an immune checkpoint, said antibody having a mutated Fc region compared with that of a parent antibody, having improved affinity for the FcgRIIIa receptor (CD16a) and/or increased ADCC activity compared with the parent antibody, said mutated Fc region comprising at least one combination of 2 following mutations:
i) a mutation selected from among 307N, 326E, 326T, 334N, 334R, 352L, 378V, 378T, 394P, 396L, 397M, 421T, 434Y and 434S; and
ii) at least one mutation selected from among 226G, 226Y, 227S, 228L, 228R, 230S, 230T, 230L, 231V, 234P, 241L, 243I, 243L, 246R, 246E, 247T, 248E, 253F, 254F, 255W, 259A, 261R, 262A, 263A, 264E, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 330V, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 389K, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434Y, 434S, 435R, 436H, 439R, 440G, 440N, 442F, 442P and 447N,
   the numbering being that of the EU index or Kabat equivalent, and provided that mutation (i) does not take place on the same amino acid as mutation (ii).

In a first embodiment, the mutated Fc region of the antibody of the invention comprises at least one combination of 2 following mutations:
i) a mutation selected from among 378V, 378T, 434Y and 434S; and
ii) at least one mutation selected from among 226G, 228L, 228R, 230S, 230T, 230L, 241L, 264E, 307P, 315D, 330V, 362R, 378V, 378T, 389T, 389K, 434Y and 434S,
the numbering being that of the EU index or Kabat equivalent, and provided that mutation (i) does not take place on the same amino acid as mutation (ii).

According to the preceding paragraph, the mutated Fc region of the antibody of the invention may also comprise an additional mutation selected from among 361D, 428L, 307A, 382V, 259I, 256N and 383N.

More preferably, the mutated Fc region of the antibody of the invention comprises the combinations of mutations selected from among N315D/A330V/N361 D/A378V/N434Y, N315D/N361 D/A378V/N434Y, P230S/N315D/M428L/N434Y, T307A/N315 D/A330V/E382V/N389T/N434Y, V259I/N315D/N434Y and T256N/A378V/S383N/N434Y.

In a second embodiment, the present invention relates to an antibody directed against a ligand of an immune checkpoint, said antibody having a mutated Fc region compared with that of a parent antibody, having improved affinity for the FcgRIIIa receptor (CD16a) and/or increased ADCC activity compared with the parent antibody, said mutated Fc region comprising at least one combination of 2 following mutations:
i) a mutation selected from among 307N, 326E, 326T, 334N, 334R, 352L, 378V, 378T, 394P, 396L, 397M and 421T; and
ii) at least one mutation selected from among 226Y, 227S, 230S, 231V, 234P, 243I, 243L, 246R, 246E, 247T, 248E, 253F, 254F, 255W, 259A, 261R, 262A, 263A, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434S, 435R, 436H, 439R, 440G, 440N, 442F, 442P and 447N,
   the numbering being that of the EU index or Kabat equivalent, and provided that mutation (i) does not take place on the same amino acid as mutation (ii).

More preferably, in this second embodiment, the mutated Fc region of the antibody of the invention comprises at least one combination of 2 following mutations:
i) a mutation selected from among 378V, 378T, 326E, 397M, 334N, 396L, 434Y and 434S; and
ii) at least one mutation selected from among 226G, 228L, 228R, 230S, 230T, 230L, 241L, 264E, 307P, 315D, 316D, 330V, 362V, 397M, 334N, 248E, 231V, 246R, 336T, 421T, 361H, 366A, 439R, 290E, 394P, 307P, 378V, 378T, 286I, 286Y, 298N, 389T, 389K, 434Y and 434S,
   the numbering being that of the EU index or Kabat equivalent, and provided that mutation (i) does not take place on the same amino acid as mutation (ii).

More preferably, the mutated Fc region of the antibody of the invention comprises at least one combination of 2 following mutations:
i) a mutation selected from among 378V, 326E, 397M, 334N and 396L; and
ii) at least one mutation selected from among 316D, 397M, 334N, 248E, 231V, 246R, 336T, 421T, 361H, 366A, 439R, 290E, 394P, 307P, 378V, 378T, 286I, 286Y and 298N,
   the numbering being that of the EU index or Kabat equivalent, and provided that mutation (i) does not take placed on the same amino acid as mutation (ii).

According to the preceding paragraph, the mutated Fc region of the antibody of the invention may also comprise an additional mutation selected from among 333G, 352S, 423Y, 315D, 412M and 366A.

More preferably, the mutated Fc region of the antibody of the invention comprises the combinations of mutations selected from among 248E/378V, 333G/378T/397M, 396L/421 T/378V, 396L/421 T, 316D/326E/378V, 298N/378V, 336T/378V, 334N/352S/397M/378V, 286I/378V/423Y, 315D/361H/396L, 231V/378V, 378T/397M/412M, 286Y/352S/378V, 290E/366A/378V, 286I/396L/421T and 334N/352S/397M. More preferably, the mutated Fc region of the antibody of the invention comprises the combination of mutations 334N/352S/397M/378V.

Preferably, the mutated Fc region of the antibody of the invention comprises at least one combination of 2 mutations according to the first embodiment, and at least one combination according to the second embodiment.

Therefore, preferably, the mutated Fc region of the antibody of the invention comprises a combination of mutations selected from among N315D/A330V/N361 D/A378V/N434Y, V259I/N315D/N434Y and N315D/N361 D/A378V/N434Y, and a combination of mutations selected from among 248E/378V, 333G/378T/397M, 396L/421T/378V, 396L/421T, 316D/326E/378V, 298N/378V, 336T/378V, 334N/352S/397M/378V, 286I/378V/423Y, 315D/361H/396L, 231V/378V, 378T/397M/412M, 286Y/352S/378V, 290E/366A/378V, 286I/396L/421T and 334N/352S/397M.

Alternatively, preferably, the mutated Fc region of the antibody of the invention comprises a combination of mutations selected from among N315D/A330V/N361 D/A378V/N434Y, V259I/N315D/N434Y, K334N/P352S/V397M/A378V et N315D/N361 D/A378V/N434Y, as well as one of the following mutations: V240M, L242K, L242G, L242F, F243L, E258R, T260A, V262A, K290G, Y296W, S298R or V302R.

Preferably, the mutated Fc region of the antibody of the invention comprises a combination of mutations selected from among DN315D/A330V/N361 D/A378V/N434Y, V259I/N315D/N434Y, K334N/P352S/V397M/A378V and N315D/N361 D/A378V/N434Y, as well as at least one of the following mutations: K290G, Y296W or N434Y.

Preferably, the mutated Fc region of the antibody of the invention comprises a combination of mutations selected from among K334N/P352S/V397M/A378V and N315D/N361D/A378V/N434Y, as well as at least one of the following mutations Y296W, N434Y or Y296W/N434Y.

Preferably, the mutated Fc region of the antibody of the invention comprises a combination of mutations selected from among Y296W/K334N/P352S/V397M/A378V, Y296W/N315D/N361 D/A378V/N434Y, K334N/P352S/V397M/A378V/N434Y and Y296W/K334N/P352S/V397M/A378V/N434Y.

By «antibody» it is meant a tetramer composed of two identical heavy chains each of 50-70 kDa (called H chains) and of two identical light chains each of 25 kDa (called L chains) bonded together by intra- and inter-chain disulfide bridges. This tetramer comprises at least two variable regions at the N-terminal end of each chain (called VL for the light chains and VH for the heavy chains) and a constant region at the C-terminal end called Fc formed of a single domain called CL for the light chain and of three or four domains for the heavy chain called CH1, CH2, CH3 and possibly CH4.

Each domain comprises about 110 amino acids and is comparably structured. The 2 heavy chains are bonded by disulfide bridges at CH2 and each heavy chain is bonded to a light chain via a disulfide bridge between CH1 and CL. The region which determines the specificity of the antibody for the antigen is carried by the variable parts and it is these parts which are responsible for recognition of the antigen. In each variable region, three loops are joined together to form a binding site to the antigen. Each of the loops is called a Complement Determining Region (CDR). As for the constant regions, these preferably bind to the Fc receptors Fc (FcR) of the effector cells. The assembling of the chains composing an antibody allows a characteristic Y-shaped three-dimensional structure to be defined where:
the base of the Y corresponds to the constant region Fc, or Fc fragment: it is recognised by the Fc receptors to mediate the effector functions of the antibody; and
the ends of the Y arms correspond to the respective assembling of the variable region of a light chain and variable region of a heavy chain, said ends forming the Fab region and determining the specificity of the antibody for the antigen.

There are five types of heavy chains (alpha, gamma, delta, epsilon, mu), which determine the classes of immunoglobulins (IgA, IgG, IgD, IgE, IgM). The light chain group comprises two sub-types, lambda and kappa. The kappa and lambda light chains are shared by all classes and sub-classes. In man, the proportion of kappa and lambda produced lies in a ratio of 2 to 1.

IgGs are the most abundant immunoglobulins in serum (75-80% of circulating antibodies). They are present in the form of monomers and have a half-life of 21 days on average.

There are four types of gamma heavy chains, which determines four IgG sub-classes (IgG1 for gamma1, IgG2 for gamma2, IgG3 for gamma3 and IgG4 for gamma4). These four sub-classes differ in the number and variable positions of the disulfide bridges (Basic and Clinical Immunology, 8^{th} Edition, Daniel P. Stites, Abba I. Terr and Tristram G. Parslow (Eds.), Appleton & Lange, Norwalk, Conn., 1994, page 71 and Chapter 6).

The four sub-classes of human IgGs also differ in biological activity, despite highly homologous structures (more than 95% sequence homology for the Fc regions).

Antibodies notably comprise full-length immunoglobulins, monoclonal antibodies, multi-specific antibodies, chimeric antibodies, humanized antibodies and fully human antibodies.

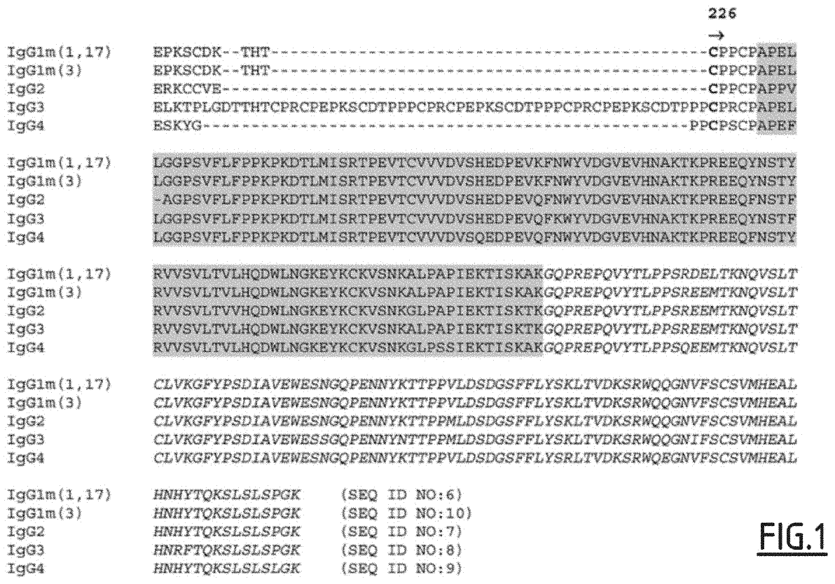

The term “Fc” or “Fc region” or “Fc fragment” designates the constant region of an antibody of total length with the exclusion of the first domain of immunoglobulin constant region (CH1-CL). Therefore, Fc refers to the two last domains (CH2 and CH3) of an IgG constant region, and to the flexible N-terminal hinge of these domains. For human IgG1, the Fc region comprises the domains CH2 and CH3 as well as the lower hinge region between CH1 and CH2. Therefore, the Fc region corresponds to the C226 residue as far as its carboxy-terminal end i.e. the residues at position 226 to 447, as per EU Index numbering or Kabat equivalent. The analogue domains for other IgG sub-classes can be determined from the alignment of amino acid sequences of the heavy chains or heavy chain fragments of IgG sub-classes with human IgG1 (see FIG. 1). The Fc region used may additionally comprise part of the upper hinge region located between positions 216 to 226 as per the EU Index or Kabat equivalent; in this case, the Fc region used corresponds to the residues at position 216 to 447, 217 to 447, 218 to 447, 219 to 447, 220 to 447, 221 to 447, 222 to 447, 223 to 447, 224 to 447 or 225 to 447, where the numbering is that of the EU Index or Kabat equivalent. Preferably in this case, the Fc region used corresponds to the residues at position 216 to 447 as per the numbering of the EU Index or Kabat equivalent.

Preferably, the Fc region used is selected from among the sequences SEQ ID NO: 1, 2, 3, 4 and 5. Preferably, the Fc region of the parent antibody has the sequence SEQ ID NO: 1. The sequences represented in SEQ ID NO: 1, 2, 3, 4 and 5 are devoid of an N-terminal hinge region.

The sequences represented in SEQ ID NO: 6, 7, 8, 9 and 10 correspond to the sequences represented in SEQ ID NO: 1, 2, 3, 4 and 5 respectively with their N-terminal hinge regions. Therefore, in one particular embodiment, the Fc region of the parent antibody is selected from among sequences SEQ ID NO: 6, 7, 8, 9 and 10.

Preferably, the Fc region of the parent antibody has a sequence corresponding to positions 1-232, 2-232, 3-232, 4-232, 5-232, 6-232, 7-232, 8-232, 9-232, 10-232 or 11-232 of sequence SEQ ID NO: 6.

«Fv fragment» designates the smallest fragment maintaining the binding properties of the antibody. It is solely composed of light chain VL and heavy chain VH variable regions, it therefore fixes the antigen with the same affinity as the whole antibody.

By «position» it is meant a position in the sequence of amino acids. For the Fc region, the positions are numbered in accordance with the EU Index or Kabat equivalent.

By «amino acid» or «residue» it is meant one of the 20 natural amino acids or natural analogues.

The term «immune checkpoints» refers to receptors located on the surface of immune effector cells capable of inhibiting (inhibitory immune checkpoints) or activating immune response (stimulatory immune checkpoints) after engaging with their ligands.

The immune checkpoint is preferably selected from among GITR, OX40, PD1, CTLA4, TIM3, LAG3, KIR, BTLA1 and a2AR.

By «activation receptor» in the present invention it is meant a surface receptor which, after interaction with its ligand, causes triggering of a signalling pathway leading to activation of immune response. The stimulatory immune checkpoint is preferably selected from among GITR and OX40.

GITR, also called tumour necrosis factor receptor superfamily member 18 (TNFRSF18) or activation-inducible TNFR family receptor (AITR), is a protein having its expression increased when the T cells are activated.

OX40, also called CD134 or Tumour necrosis factor receptor superfamily, member 4 (TNFRSF4), is a protein which is not constitutively expressed on naïve T cells. It is expressed when the latter are activated. Its ligand OX40L is also expressed on activated antigen presenting cells.

By «inhibitory receptor» in the present invention it is meant a surface receptor which, after interaction with its ligand, causes triggering of a signalling pathway leading to inactivation of immune response.

Preferably, the immune checkpoint is inhibitory. More preferably, it is selected from among PD1, CTLA4, TIM3, LAG3, KIR, BTLA1 and a2AR.

PD1 (Programmed cell Death factor 1) is an inhibitory receptor of the CD28 family expressed on the surface of activated T and B lymphocytes and Natural Killers. Its role is to limit the activity of the effector cells in secondary lymphoid tissues or tumours, thereby imparting it with a major tumour resistance mechanism. PD1 inhibits lymphocyte functions when it is engaged with one of its ligands, PDL1 (or B7-H1 or CD274) or PDL2. PDL1 is a molecule expressed on the surface of tumour cells. In the event of chronic exposure to the PDL1 ligand (e.g. the case with cancer), the expression of PD1 on the surface of effector cells is increased, leading to a phenomenon of anergy. Anti-PD1 antibodies are used in the treatment of cancers such as lung cancer, non-small cell lung cancer (NSCLC), mesotheliomas, bladder cancer, colorectal cancer, metastatic colorectal cancer, bladder cancer, breast cancer, head and neck cancers, testicular cancer, endometrial cancer, oesophageal cancer, thymus cancer, haematological cancer, advanced haematological cancer such as non-Hodgkin's lymphoma, Hodgkin's lymphoma, chronic lymphoid leukaemia, multiple melanomas, acute myeloid leukaemia, brain tumours, glioblastomas, solid tumours, gastric adenocarcinomas, germ cell tumours, hepatocellular carcinoma, melanomas, metastatic melanomas, lymphomas, diffuse large B-cell lymphomas (DLBCL), follicular lymphomas, non-resectable or metastatic melanomas, or advanced renal cell carcinoma.

CTLA4 (Cytotoxic T-lymphocyte-associated antigen 4) is an inhibitory receptor solely expressed on the surface of T lymphocytes. Its role is to regulate the first activation steps of T lymphocytes. 48 hours after activation of the T lymphocytes via their receptor (T Cell Receptor—TCR), CTLA4 engages with its ligands (CD80 or CD86) expressed on the surface of the antigen presenting cells (APCs) at the lymph nodes and sometimes at tumours. This causes a signalling cascade leading to inhibition of the T lymphocytes. Anti-CTLA4 antibodies are used in the treatment of cancers such as lung cancer, non-small cell lung cancer (NSCLC), small cell lung carcinoma, breast cancer, pancreatic cancer, prostate cancer, gastric cancer, renal cancer, head and neck cancer, liver cancer, metastatic or non-resectable melanomas, skin melanoma with lymph node involvement, renal carcinoma, myelomas, lymphomas, hepatocellular carcinoma, brain metastases, solid tumours, mesotheliomas, lymphomas or melanomas.

TIM3 (T-cell immunoglobulin and mucin-domain containing-3) is a receptor expressed on the surface of IFN γ-secreting T lymphocytes. One of its ligands is galectine-9, a protein overexpressed in tumour cells. The engaging of TIM3 with galectine-9 leads to inhibition of immune response.

LAG3 (lymphocyte activation gene 3) also called CD223 is a molecule expressed on the surface of T lymphocytes. Its only known ligand is the class II Major Histocompatibility Complex (MHC II) which can be overexpressed in some cancers but also by antigen presenting cells (APC) (macrophages and dendritic cells) infiltrated at tumours. The engaging of LAG3 with its receptor causes an anergy phenomenon.

KIR (Killer-cell immunoglobulin-like receptor) is a molecule expressed on the surface of Natural Killers, T lymphocytes and APCs. When KIR binds to its ligand, the class I MHC (MHC I), the effector response of the Natural Killers is attenuated at tumour sites.

BTLA1 (B and T lymphocyte attenuator), also called CD272, is a molecule expressed on the surface of lymphocytes. Its ligand, the HVEM molecule (herpesvirus entry mediator), is expressed in some types of tumours (notably the case in melanomas).

a2ARs are expressed in different types of immune effector cells, in particular in T lymphocytes and in endothelial cells. When a2AR binds to its ligand adenosine (which accumulates in tumours), the CD4+ cells express FOXP3 and differentiate into regulatory T cells, consequently leading to inhibition of immune response.

Preferably, the ligand of the immune checkpoint is selected from among OX40L, PDL1, PDL2, CD80, CD86, galectine-9, MHC II, MHC I, HVEM and adenosine.

More preferably, the immune checkpoint is selected from among PD1 and CTLA4.

More preferably, the immune checkpoint is PD1. Therefore, preferably, the anti-immune checkpoint antibody of the invention is an anti-PD1 or anti-CTLA4 antibody. More preferably, the anti-immune checkpoint antibody is an anti-PD1 antibody.

Preferably, the ligand is selected from among PDL1, PDL2, CD80 and CD86. Preferably, the ligand is PDL1 or PDL2, preferably PDL1. Therefore, preferably, the anti-ligand antibody of the invention is an anti-PDL1, anti-PDL2, anti-CD80 or anti-CD86 antibody. Preferably, the anti-ligand antibody of the invention is an anti-PDL1 antibody.

The anti-PDL1 antibody of the invention may comprise a variable region corresponding to the sequence of an Fv fragment of a known anti-PDL1 antibody e.g. the atezolizumab antibody, durvalumab antibody or avelumab antibody. Therefore, the anti-PDL1 antibody of the invention may comprise a light chain variable sequence (VL) and a heavy chain variable sequence (VH) corresponding to the VL and VH sequences of the atezolizumab antibody, durvalumab antibody or avelumab antibody respectively.

The sequences are the following:
the VH and VL sequences of the atezolizumab antibody are the sequences SEQ ID NO:11 and 12 respectively;
the VH and VL sequences of the durvalumab antibody are the sequences SEQ ID NO:13 and 14 respectively; and
the VH and VL sequences of the avelumab antibody are the sequences SEQ ID NO:15 and 16 respectively.

Therefore, preferably, the anti-PDL1 antibody of the invention comprises a VH of sequence SEQ ID NO:11 and a VL of sequence SEQ ID NO:12.

Alternatively, preferably, the anti-PDL1 antibody of the invention comprises a VH of sequence SEQ ID NO:13 and a VL of sequence SEQ ID NO:14.

Alternatively, preferably, the anti-PDL1 antibody of the invention comprises a VH of sequence SEQ ID NO:15 and a VL of sequence SEQ ID NO:16.

The term «parent antibody» is used to define the reference antibody which can be of natural or synthetic origin. In the context of the present invention, the parent antibody comprises an Fc region called «parent Fc region». Said parent Fc region is selected from the group of wild-type Fc regions and fragments thereof. By «wild-type» (WT), it is meant here a sequence of amino acids or nucleotide sequence found in nature i.e. which is of natural origin, including allelic variations, and which has not been intentionally modified via molecular biology techniques such as mutagenesis. For example, the Fc regions of «wild-type» particularly refer to the Fc region of IgG1 having the sequence SEQ ID NO:1 (allotype G1m1,17), the Fc region of IgG2 having the sequence SEQ ID NO: 3, the Fc region of IgG3 having the sequence SEQ ID NO: 4, the Fc region of IgG4 having the sequence SEQ ID NO: 5, and the Fc region of IgG1 having the sequence SEQ ID NO: 1 (allotype G1m3). The Fc regions of «wild-type» also refer to the Fc regions corresponding to sequences SEQ ID NO: 6 to SEQ ID NO: 10. Preferably, the parent antibody comprises a parent Fc region which is a human Fc region, preferably an Fc region of human IgG1 or human IgG2. The parent antibody may also comprise modifications of amino acids pre-existing in the Fc region (e.g. an Fc mutant) compared with the Fc regions of wild-type.

By «immune effector cells», it is meant cells which carry out the immune mechanism and express an Fc receptor (FcR). Those cells notably considered as effector cells are lymphocytes including Natural Killer cells (NK), macrophages, monocytes, neutrophils, eosinophils, basophils, mastocytes, dendritic cells including Langerhans cells and platelets.

By «mutation» it is meant a change of at least one amino acid in the sequence of a polypeptide, in particular a change of at least one amino acid in the Fc region of the parent antibody. The antibody obtained then comprises a mutated Fc region compared with that of the parent antibody. Preferably, mutation is a substitution, insertion or deletion of at least one amino acid at a particular position. The mutated Fc regions can have several mutations, affecting several amino acids, preferably two to ten.

By «substitution» it is meant the replacement of an amino acid by another amino acid at a particular position in a sequence of the parent antibody. For example, substitution 434S refers to a variant antibody (or mutant), here a variant for which an amino acid at position 434 is replaced by serine. Preferably, the following mutation wording is used: «434S» or «N434S», and means that the parent antibody comprises asparagine at position 434, which is replaced by serine in the variant. In the event of a combination of substitutions, the preferred format is the following: «259I/315D/434Y» or «V259I/N315D/N434Y». This means that there are three substitutions in the variant, at positions 259, 315 and 434, and that the amino acid at position 259 of the parent antibody, namely valine, is replaced by isoleucine, that the amino acid at position 315 of the parent antibody, namely asparagine, is replaced by aspartic acid, and that the amino acid at position 434 of the parent antibody, namely asparagine, is replaced by tyrosine.

By «deletion of amino acids» or «deletion», it is meant the suppression of an amino acid at a particular position in a sequence of the parent antibody. For example, E294del or 294del designates the suppression of glutamic acid at position 294.

By «insertion of amino acid» or «insertion», it is meant the addition of an amino acid at a particular position in a sequence of the parent antibody. For example, the insertion G>235-236 designates an insertion of glycine between positions 235 and 236.

A mutated Fc variant of the invention can be generated by any well-known mutagenesis method. For example, via overlap extension PCR using two sets of primers adapted to integrate the targeted mutation(s) with the codon(s) encoding the desired amino acid. Alternatively, de novo synthesis of genes containing the nucleotide sequence comprising the mutations of interest, can be used.

Throughout the present application, the numbering of the residues in the Fc region is that of the immunoglobulin heavy chain in accordance with the EU Index or Kabat equivalent in Kabat et al. (Sequences of Proteins of Immunological Interest, 5^{th} Ed. Public Health Service, National Institutes of Health, Bethesda, Md., 1991). The expression «EU Index or Kabat equivalent» refers to the EU numbering of the residues of the human IgG1, IgG2, IgG3 or IgG4 antibody as given on the IMGT website: http://www.imgt.org/IMGTScientificChart/Numbering/Hu_IGHGnber.html).

The affinity of the mutated Fc region of the antibody of the invention for the FcgRIIIa receptor (CD16a) is increased compared with that of the parent antibody.

Preferably, this affinity is improved compared with that of the parent antibody, by a ratio of at least 2, preferably higher than 5, preferably higher than 10, preferably higher than 15, preferably higher than 20, preferably higher than 25, preferably higher than 30.

By «improved affinity for the FcgRIIIa receptor (CD16a)», it is meant an increase in the in vivo or in vitro binding affinity of the mutated Fc of the invention for the FcgRIIIa receptor (CD16a) compared with the parent antibody. The FcgRIIIa receptor (CD16a) is involved in ADCC and has V/F polymorphism at position 158.

Preferably, the mutated Fc region of the antibody of the invention also has modified affinity, compared with that of the parent antibody, for at least one of the following receptors: the C1q complement component, FcgRIIa (CD32a) and FcgRIIb (CD32b). The C1q complement component is involved in complement-dependent cytotoxicity (CDC). The FcgRIIa receptor (CD32a) is involved in platelet activation and phagocytosis; it has H/R polymorphism at position 131.

Preferably, the mutated Fc region of the antibody of the invention has increased affinity, compared with that of the parent antibody, for at least one of the following receptors: C1q complement component, FcgRIIa (CD32a) and FcgRIIb (CD32b). By «increased affinity» for a receptor, it is meant an increase in the binding affinity, in vivo or in vitro, of the mutated Fc region of the invention for said receptor compared with the parent antibody. In this case, this affinity is improved, compared with that of the parent antibody, by a ratio of at least 2, preferably higher than 5, preferably higher than 10, preferably higher than 15, preferably higher than 20, preferably higher than 25, preferably higher than 30.

Preferably, the mutated Fc region of the antibody of the invention also has affinity for the C1q complement that is increased compared with that of the parent antibody. Therefore, preferably, the antibody of the invention has increased CDC activity compared with that of the parent antibody.

The affinity of an antibody for an FcR can be assessed using methods well-known in the prior art. For example, persons skilled in the art can determine affinity (Kd) using surface plasmon resonance (SPR). Alternatively, skilled persons can conduct an appropriate ELISA assay. An appropriate ELISA assay allows comparison between the binding forces of the parent Fc and mutated Fc. The detected signals specific to the mutated Fc and parent Fc are compared.

The present invention also relates to a composition comprising antibodies directed against a ligand of an immune checkpoint, having a modified Fc fragment compared with that of a parent antibody, and having improved affinity for CD16a and/or increased ADCC activity compared with the parent antibody, wherein said modified Fc fragments have N-glycans on their glycosylation site, said N-glycans having a fucosylation level of less than than 65%, preferably less than 60%, preferably less than 55%, preferably less than 50%, further preferably less than 45%, preferably less than 40%, preferably less than 35%, preferably less than 30%, preferably less than 25%, preferably less than 20%. This composition is called «composition of the invention».

By level of fucosylation, it is meant the ratio of N-glycans present on the Fc fragments having a fucose residue, compared with the total quantity of N-glycans of the Fc fragments in an antibody composition.

Preferably, said antibody composition comprises a single type of antibody comprising a mutated Fc region. In other words, the composition comprises antibody molecules of same sequence.

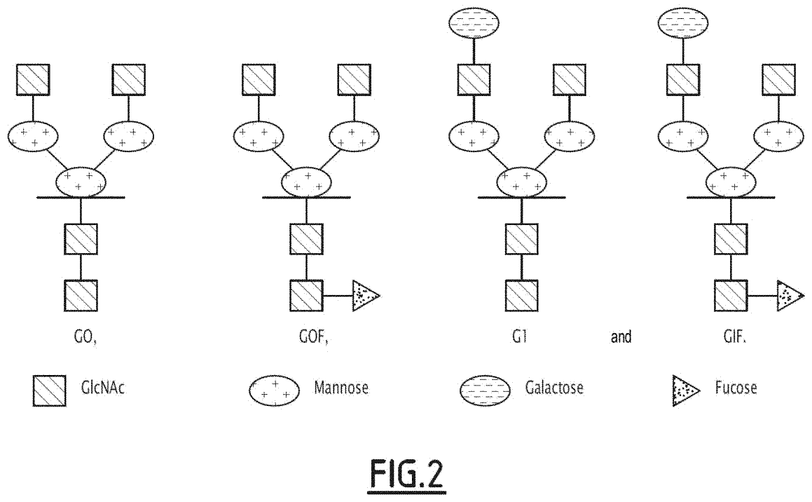

Preferably, the antibody composition of the invention is scarcely fucosylated. By «scarcely fucosylated» it is meant a composition which comprises antibodies in which the Fc fragments have N-glycans on their glycosylation site (Asn 297) which have a fucosylation level of less than 65%, preferably less than 60%, preferably less than 55%, preferably less than 50%, further preferably less than 45%, preferably less than 40%, preferably less than 35%, preferably less than 30%, preferably less than 25%, preferably less than 20%. Said N-glycans preferably have glycan structures of biantennary type with short chains and low sialylation. Preferably, the glycan structure has non-intercalated terminal GlcNAcs (N-Acetylglucosamine). Preferably, the glycan structure is selected from among the forms, G0F, G1 and G1F such as shown in FIG. 2.

Therefore, preferably, said N-glycans have glycan structures of biantennary type, with short chains, low sialylation, non-intercalated terminal GlcNAcs. More particularly, the antibody composition has a sialic acid content of less than 25%, 20%, 15%, or 10%, preferably 5%, 4% 3% or 2%. By sialylation percentage, it is meant the ratio of N-glycans present on the Fc fragments having a sialic acid residue, compared with the total quantity of N-glycans of the Fc fragments in an antibody composition.

One preferred antibody composition of the invention has a content higher than 60%, preferably higher than 80% for the forms G0+G1+G0F+G1F, on the understanding that the content of the forms G0F+G1F is less than 50%, preferably less than 40%, preferably less than 30%. Preferably, the N-glycan content is higher than 60% for the forms G0+G1+G0F+G1F, the fucose content being less than 65%.

The antibodies of the invention directed against a ligand of an immune checkpoint can be prepared using any method well-known in the prior art. Once the encoding nucleic acids thereof have been obtained, the antibodies of the invention can be prepared using any method known in the art.

In one embodiment, the nucleic sequences can be cloned in host cells and then expressed. The nucleic sequences can also be incorporated in an expression vector. A wide variety of suitable host cell lines can be used including, but not limited thereto, mammalian cells, bacteria, insect cells and yeasts.

The host cells, as nonlimiting examples, can be YB2/0 (ATCC, CRL-1662), SP2/0, YE2/0, PERC6 cells, CHO cell lines, in particular CHO-K-1, CHOS, CHO-LeclO, CHO-Lecl, CHO-Lecl3, CHO Pro-5, CHO dhfr-, Wil-2, Jurkat, Vero, COS-7, HEK particularly 293-HEK, BHK, KGH6, NSO, SP2/0-Ag 14, P3X63Ag8.653, C127, JC, LA7, ZR-45-30, hTERT, NM2C5 or UACC-812. In one preferred embodiment of the invention, the antibody is expressed in the YB2/0 cell.

Alternatively, the host cells can be cells of a non-human transgenic organism, in particular cells of transgenic animals modified to produce the antibody in their milk, or else cells of transgenic plants modified to produce the antibody. With respect to cells of transgenic animals modified to produce the antibody in their mik, the expression of DNA sequences encoding the antibody of the invention, directed against a ligand of an immune checkpoint, is controlled by a mammalian casein promoter or mammalian whey promoter, said promoter not naturally controlling transcription of said gene, and the DNA sequences also containing a secretion sequence of the protein. The secretion sequence comprises a secretion signal interpositioned between the coding sequence and the promoter. The animal can be selected for example from among sheep, goats, does, ewes or cows.

In this embodiment, preferably whole anti-PDL1 IgG1s mutated according to the invention can be generated by production in the milk of a transgenic animal e.g. a transgenic goat, and purified by extracting the milk. For this purpose, the sequence encoding the heavy chain and the sequence encoding the light chain are prepared in an expression vector under the control of a promoter specific to the mammary glands e.g. a mammalian casein promoter allowing the production and secretion of the antibody to be directed into the milk of the mammary glands. Said method is particularly described in application EP0741515.

The present invention also relates to products (hereafter «products of the invention») containing:

a) an antibody of the invention directed against a ligand of an immune checkpoint, or a composition of the invention, and

b) an antibody directed against an immune checkpoint, having a modified Fc fragment compared with that of a parent antibody, having improved affinity for the FcRn receptor and optionally reduced functional activity mediated by the Fc region, said immune checkpoint being selected from among PD1, CTLA4, TIM3, LAG3, KIR, BTLA1 and a2AR, as combination products for simultaneous, separate or time-staggered administration, for use thereof in the prevention or treatment of cancers.

This anti-immune checkpoint antibody b) particularly allows binding to the target immune cell. For example, by binding to the receptor present on the T lymphocytes infiltrating tumours (e.g. PD1), it prevents binding between PD1 (present on T lymphocytes) and PDL1 (present on tumour cells). The antibody is then termed a neutralising antibody.

The anti-immune checkpoint antibody b) has a modified Fc fragment imparting thereto greater affinity for the FcRn receptor.

The FcRn receptor corresponding to the «neonatal Fc receptor» is a protein composed of a heavy chain encoded by the FcRn gene (called FCGRT in Man) and of a light chain, the molecule of β2-microglobulin. It can bind the Fc region of IgGs and has the characteristic of increasing the half-life of IgGs which attach thereto. FcRn can be found in different organisms including, but not limited thereto, humans, mice, rats, rabbits and monkeys.

By «greater affinity for FcRn» it is meant increased binding affinity, in vivo or in vitro, of the mutated Fc region of the invention for FcRn, compared with that of the parent antibody. The capability of the mutated Fc region of the invention to bind with a FcRn receptor can be assessed in vitro by ELISA assay, as described for example in patent application WO2010/106180. Increased binding to FcRn translates as an improvement in serum retention in vivo et, and hence an increase in half-life.

Preferably, the anti-immune checkpoint antibody b) (having greater affinity for the FcRn receptor) comprises at least two mutations, said mutations being selected from among:
(i) a modification selected from among 378V, 378T, 434Y and 434S; and
(ii) at least one modification selected from among 226G, P228L, P228R, 230S, 230T, 230L, 241 L, 264E, 307P, 315D, 330V, 362R, 378V, 378T, 389T, 389K, 434Y and 434S,
   the numbering being that of the EU Index or Kabat equivalent, and provided that mutation (i) does not take place on the same amino acid as mutation (ii).

Preferably, the anti-immune checkpoint antibody b) has an Fc region comprising at least one combination of mutations selected from among 226G/315D/434Y, 230S/315D/434Y, 230T/315D/434Y, 230T/264E/434S, 230T/389T/434S, 241 L/264E/378V, 241L/264E/434S, 250A/389K/434Y, 259I/315D/434Y, 284E/378T/396L, 264E/378V/434Y, 345D/330V/434Y, 315D/382V/434Y and 378V/383N/434Y compared with the Fc region of said parent antibody, the numbering being that of the EU Index or Kabat equivalent.

Preferably, the anti-immune checkpoint antibody b) has an Fc region comprising at least one mutation selected from among 226G, 227L, 230S, 230T, 230L, 231T, 241L, 243L, 250A, 256N, 259I, 264E, 265G, 267R, 290E, 294del, 303A, 305A, 307P, 307A, 308I, 315D, 322R, 325S, 327V, 330V, 342R, 347R, 352S, 361D, 362R, 362E, 370R, 378V, 378T, 382V, 383N, 386R, 386K, 387T, 389T, 389K, 392R, 395A, 396L, 397M, 403T, 404L, 415N, 416K, 421T, 426T, 428L, 433R, 434Y, 434S and 439R compared with the Fc region of said parent antibody, the numbering being that of the EU Index or Kabat equivalent.

Preferably, the anti-immune checkpoint antibody b) has an Fc region comprising a combination of mutations selected from among 307A/315D/330V/382V/389T/434Y, 256N/378V/383N/434Y, 345D/330V/361 D/378V/434Y, 259I/315D/434Y, 230S/315D/428L/434Y, 241 L/264E/307P/378V/433R, 250N389K/434Y, 305A/315D/330V/395N343Y, 264E/386R/396L/434S/439R, 315D/330V/362R/434Y, 294del/307P/434Y, 305N315D/330V/389K/434Y, 315 D/327V/330V/397M/434Y, 230T/241 L/264E/265G/378V/421 T, 264E/396U415N/434S, 227L/264E/378V/434S, 264E/378T/396L, 230T/315D/362R/426T/434Y, 226G/315D/330V/434Y, 230L/241 L/243L/264E/307P/378V, 250A/315D/325S/330V/434Y, 290E/315D/342R/382V/434Y, 241 L/315D/330V/392R/434Y, 241 L/264E/307P/378W/434S, 230T/264E/403T/434S, 264E/378V/416K, 230T/315D/362E/434Y, 226G/315D/434Y, 226G/315D/362R/434Y, 226G/264E/347R/370R/378V/434S, 308I/315D/330V/382V/434Y, 230T/264E/378V/434S, 231T/241 L/264E/378T/397M/434S, 230L/264E/378W/434S, 230T/315D/330V/386K/434Y, 226G/315D/330V/389T/434Y, 267R/307P/378V/421 T/434Y, 230S/315D/387T/434Y, 230S/264E/352S/378V/434S and 230T/303A/322R/389T/404U434S compared with said parent antibody, the numbering being that of the EU Index or Kabat equivalent.

Preferably, the anti-immune checkpoint antibody b) has an Fc region comprising the combinations of mutations selected from among N315D/A330V/N361 D/A378V/N434Y, P230S/N315D/M428L/N434Y, E294del/T307P/N434Y, T307A/N315 D/A330V/E382V/N389T/N434Y, V259I/N315D/N434Y, V259I/E294Del/N315D/N434Y and T256N/A378V/S383N/N434Y.

The anti-immune checkpoint antibody b) can have reduced functional activity mediated by the Fc region, compared with that of the parent antibody.

By «functional activity mediated by the Fc region» it is meant the effector functions mediated by the Fc region. Included in said functional activities mediated by the Fc region are antibody-dependent cell-mediated cytotoxicity (ADCC), complement-dependent cytotoxicity (CDC), antibody-dependent cell phagocytosis (ADCP), endocytosis activity, cytokine secretion or a combination of at least two of these activities. Preferably, the functional activity mediated by the Fc region under consideration in the invention is ADCC. This functional activity can be evaluated with methods well known in the prior art.

In particular, the functional activity mediated by the Fc region is reduced compared with that of the parent antibody, by a ratio of at least 2, preferably higher than 5, preferably higher than 10, preferably higher than 15, preferably higher than 20, preferably higher than 25, preferably higher than 30.

The mutated Fc region of the antibody b) of the invention preferably has reduced affinity for at least one of the receptors of the Fc region (FcR) selected from among C1q complement and the receptors FcgRIIIa (CD16a), FcgRIIa (CD32a) and FcgRIIb (CD32b). The receptors of the Fc region that are involved are:
C1q involved in CDC activity,
FcgRIIIa receptor (CD16a) involved in ADCC and has V/F polymorphism at position 158,
FcgRIIa receptor (CD32a) involved in platelet activation and phagocytosis, and has H/R polymorphism at position 131, and
FcgRIIb receptor (CD32b) involved in inhibition of cell activity.

The affinity of an antibody for a FcR can be evaluated with methods well known in the prior art. For example, persons skilled in the art can determine affinity (Kd) using surface plasmon resonance (SPR). Alternatively, skilled persons can conduct an appropriate ELISA assay. An appropriate ELISA assay allows comparison between the binding forces of the parent Fc and the mutated Fc. The detected signals specific to the mutated Fc and parent Fc are compared.

When the anti-immune checkpoint antibody b) has reduced functional activity mediated by the Fc region, it allows neutralisation of the binding between the immune checkpoint and its ligand (e.g. PD1 and PDL1), without having effector activity. It thereby allows blockading of the immune checkpoint.

Preferably, the anti-immune checkpoint antibody b) has an Fc region at least having the mutation del294.

Preferably, in another embodiment, the anti-immune checkpoint antibody b) is aglycosylated. For example, it can be mutated on asparagine 297 by an amino acid such as alanine preventing glycosylation. Therefore, preferably, the anti-immune checkpoint antibody b) has a mutated Fc region having mutation N297A compared with the parent antibody.

Preferably, the products of the invention contain:

a) an antibody of the invention directed against PDL1, or a composition of the invention comprising antibodies directed against PDL1, and

b) an antibody directed against PD1, having a modified Fc fragment compared with that of a parent antibody, having improved affinity for the FcRn receptor, and optionally reduced functional activity mediated by the Fc region,

as combination products for simultaneous, separate or time-staggered administration, for use thereof in the prevention or treatment of cancers.

The antibody directed against PD1 (antibody b) is such as described in the foregoing and preferably has at least the mutation del294.

Preferably, when the anti-immune checkpoint antibody b) is an anti-PD1 antibody, it comprises a variable region corresponding to the sequence of an Fv fragment of a known anti-PD1 antibody, for example the nivolumab antibody or pembrolizumab antibody. Therefore, the anti-PD1 antibody of the invention may comprise a light chain variable sequence (VL) and a heavy chain variable sequence (VH) corresponding to the VL and VH sequences of the nivolumab antibody or pembrolizumab antibody respectively.

The sequences are the following:
the VH and VL sequences of the nivolumab antibody are the sequences SEQ ID NO: 17 and 18 respectively; and
the VH and VL sequences of the pembrolizumab antibody are the sequences SEQ ID NO: 19 and 20 respectively.

Therefore, preferably, the anti-PD1 antibody of the invention comprises a VH of sequence SEQ ID NO: 17 and a VL of sequence SEQ ID NO: 18.

Alternatively, preferably, the anti-PD1 antibody of the invention comprises a VH of sequence SEQ ID NO: 19 and a VL of sequence SEQ ID NO: 20.

The invention also concerns a method for treating cancers, which comprises the administering to a patient of an anti-ligand antibody (preferably anti-PDL1) of the invention, or a composition of the invention (preferably a composition of anti-PDL1 antibodies).

Any route of administration can be envisaged, in particular parenteral routes such as the intravenous, intramuscular, sub-cutaneous, intradermal, topical routes, or via mucosal route e.g. by inhalation. The enteral (oral, rectal) and intrathecal routes are also possible. Preferably, the intravenous route is used.

The antibodies of the invention are generally formulated in pharmaceutical compositions comprising pharmaceutically acceptable excipients.

The invention also relates to a pharmaceutical composition comprising (i) at least one anti-immune checkpoint antibody of the invention, or a composition of the invention, or products of the invention and (ii) at least one pharmaceutically acceptable excipient.

By «pharmaceutical composition» it is meant a composition having curative or preventive properties with regard to human or animal diseases.

A further subject of the invention is the use of an anti-ligand antibody of the invention (preferably anti-PDL1), or of a composition of the invention (preferably of anti-PDL1 antibodies), or of products of the invention or of a pharmaceutical composition such as described in the preceding paragraph, to treat cancers.

The pharmaceutical compositions can be in any galenic form adapted to the chosen route of administration.

Preferably, they contain a pharmaceutically acceptable excipient for a formulation that can be injected. In particular this may concern isotonic, sterile formulas, saline solutions or freeze-dried compositions which, when sterilised water or physiological saline is added thereto accordingly, allow the forming of injectable solutes.

The pharmaceutical forms suitable for injection comprise sterile aqueous solutions or dispersions, oily formulations, and sterile powders for extemporaneous preparation of sterile injectable solutions or dispersions. In all cases, the form must be sterile, and must be fluid insofar as it is to be injected via syringe. It must be stable under production and storage conditions and must be protected against contaminating action of microorganisms such as bacteria and fungi.

The dispersions of the invention can be prepared in glycerol, liquid polyethylene glycols or mixtures thereof, or in oils. Under normal conditions of storage and use, these preparations contain a preserving agent to prevent the growth of microorganisms.

The pharmaceutically acceptable excipient can be a solvent or dispersion medium. Suitable fluidity can be maintained for example through the use of a surfactant. The prevention of action by microorganisms can be obtained via various antibacterial and antifungal agents. In many cases it will be preferable to include isotonic agents. Extended absorption of the injectable compositions can be obtained through the use of absorption-delaying agents in the compositions.

The sterile injectable solutions are prepared by incorporating the active substances in required amount in the suitable solvent with several of the other ingredients listed above, optionally followed by filter sterilisation. As a general rule, dispersions are prepared by incorporating the various sterilised active ingredients in a sterile excipient which contains the basic dispersion medium and other required ingredients among those listed above. Regarding sterile powders for the preparation of sterile injectable solutions, the preferred preparation methods are vacuum drying and freeze drying. When formulated, the solutions are to be administered in a manner compatible with the dosage formulation and in a therapeutically effective amount. The formulations are easily administered in a variety of galenic forms such as the injectable solutions described above, but drug release capsules and similar can also be used. For parenteral administration in an aqueous solution for example, the solution must be suitably buffered and the liquid diluent made isotonic with sufficient saline solution or glucose. These particular aqueous solutions are especially suitable for intravenous, intramuscular, subcutaneous and intraperitoneal administration. In this respect, the sterile aqueous media able to be used are known to skilled persons.

The therapeutically effective dose specific to a particular patient will depend on a variety of factors, including the disorder being treated and seriousness of the disease, the activity of the specific compound employed, the specific composition used, the age, body weight, general health, gender and food diet of the patient, the time of administration, route of administration, excretion rate of the specific compound used, length of treatment, or parallel medication.

### Indications

The immune system normally recognises tumour cells as foreign elements, which consequently leads to an antitumor immune response. However, it happens that cancer cells set up escape strategies from the immune system; they are therefore no longer recognised or eliminated by the immune system. The immune checkpoints expressed on the surface of immune effector cells form a major pathway for tumour escape. These molecules, when they are inhibitory (the case for example with PD1, CTLA4, LAG3, TIM3, KIR, BTLA1 and a2AR), have the function of attenuating immune response when they engage with their ligands which are often expressed by tumour cells.

By «cancer» it is meant any physiological condition characterised by abnormal cell proliferation.

The antibodies of the invention, the compositions of the invention, the products and the pharmaceutical composition of the invention are therefore used to treat different types of cancers. Examples of cancers notably include non-small cell lung cancer (NSCLC), non-resectable or metastatic melanomas, advanced renal cell carcinomas, bladder cancer, kidney cancer, melanomas, lung cancers, lymphomas, mesotheliomas, colorectal cancer, metastatic colorectal cancer, breast cancer, gastric cancer, head and neck cancers, brain tumours, glioblastomas, solid tumours, endometrial cancers, oesophageal cancers, gastric adenocarcinomas, germ cell tumours, testicular cancer, hepatocellular carcinoma, thymus cancer, diffuse large B-cell lymphomas (DLBCL), haematoiogical cancers, advanced haematological cancers (such as non-Hodgkin's lymphoma, Hodgkin's lymphoma, chronic lymphoid leukaemia, multiple melanomas, acute myeloid leukaemia), astrocytoma, uveal melanoma, solid sarcomas, cancers of ovarian epithelium, primary peritoneal cancers, cancers of the Fallopian tubes, cervical cancer, anal cancer, ovarian cancer, urogenital cancer, urothelial cancer, genitourinary cancer, urogenital neoplasms, thoracic tumours, adrenocortical carcinomas, biliary cancer, follicular lymphomas, pancreatic cancer, prostate cancer, cerebral metastases, liver cancer, cervical adenocarcinomas, gastro-intestinal stromal tumours, metastatic brain cancers, Merkel cell carcinomas, synovial sarcoma, fibrosarcoma, this list not being exhaustive.

The list of sequences described in the present application is the following:

| SEQ ID | | |
|---|---|---|
| NO: | Definition | Sequence |
| 1 | Fc of IgG1 | CPPCPAPELLGGPSVFLFPP |
| | G1m1, 17 | KPKDTLMISRTPEVTCVVVD |
| | | VSHEDPEVKFNWYVDGVEVH |
| | | NAKTKPREEQYNSTYRVVSV |
| | | LTVLHQDWLNGKEYKCKVSN |
| | | KALPAPIEKTISKAKGQPRE |
| | | PQVYTLPPSRDELTKNQVSL |
| | | TCLVKGFYPSDIAVEWESNG |
| | | QPENNYKTTPPVLDSDGSFF |
| | | LYSKLTVDKSRWQQGNVFSC |
| | | SVMHEALHNHYTQKSLSLSPGK |
| 2 | Fc of IgG2 | CPPCPAPPVAGPSVFLFPPK |
| | | PKDTLMISRTPEVTCVVVDV |
| | | SHEDPEVQFNWYVDGVEVHN |
| | | AKTKPREEQFNSTFRVVSVL |
| | | TVVHQDWLNGKEYKCKVSNK |
| | | GLPAPIEKTISKTKGQPREP |
| | | QVYTLPPSREEMTKNQVSLT |
| | | CLVKGFYPSDIAVEWESNGQ |
| | | PENNYKTTPPMLDSDGSFFL |
| | | YSKLTVDKSRWQQGNVFSCS |
| | | VMHEALHNHYTQKSLSLSPGK |
| 3 | Fc of IgG3 | CPRCPAPELLGGPSVFLFPP |
| | | KPKDTLMISRTPEVTCVVVD |
| | | VSHEDPEVQFKWYVDGVEVH |
| | | NAKTKPREEQYNSTFRVVSV |
| | | LTVLHQDWLNGKEYKCKVSN |
| | | KALPAPIEKTISKTKGQPRE |
| | | PQVYTLPPSREEMTKNQVSL |
| | | TCLVKGFYPSDIAVEWESSG |
| | | QPENNYNTTPPMLDSDGSFF |
| | | LYSKLTVDKSRWQQGNIFSC |
| | | SVMHEALHNRFTQKSLSLSPGK |
| 4 | Fc of IgG4 | CPSCPAPEFLGGPSVFLFPP |
| | | KPKDTLMISRTPEVTCVVVD |
| | | VSQEDPEVQFNWYVDGVEVH |
| | | NAKTKPREEQFNSTYRVVSV |
| | | LTVLHQDWLNGKEYKCKVSN |
| | | KGLPSSIEKTISKAKGQPRE |
| | | PQVYTLPPSQEEMTKNQVSL |
| | | TCLVKGFYPSDIAVEWESNG |
| | | QPENNYKTTPPVLDSDGSFF |
| | | LYSRLTVDKSRWQEGNVFSC |
| | | SVMHEALHNHYTQKSLSLSLGK |
| 5 | Fc of IgG1 G1m3 | CPPCPAPELLGGPSVFLFPP |
| | | KPKDTLMISRTPEVTCVVVD |
| | | VSHEDPEVKFNWYVDGVEVH |
| | | NAKTKPREEQYNSTYRVVSV |
| | | LTVLHQDWLNGKEYKCKVSN |
| | | KALPAPIEKTISKAKGQPRE |
| | | PQVYTLPPSREEMTKNQVSL |
| | | TCLVKGFYPSDIAVEWESNG |
| | | QPENNYKTTPPVLDSDGSFF |
| | | LYSKLTVDKSRWQQGNVFSC |
| | | SVMHEALHNHYTQKSLSLSPGK |
| 6 | Fc of IgG1 | EPKSCDKTHTCPPCPAPELL |
| | G1m1, 17 | GGPSVFLFPPKPKDTLMISR |
| | (with hinge | TPEVTCVVVDVSHEDPEVKF |
| | region) | NWYVDGVEVHNAKTKPREEQ |
| | | YNSTYRVVSVLTVLHQDWLN |
| | | GKEYKCKVSNKALPAPIEKT |
| | | ISKAKGQPREPQVYTLPPSR |
| | | DELTKNQVSLTCLVKGFYPS |
| | | DIAVEWESNGQPENNYKTTP |
| | | PVLDSDGSFFLYSKLTVDKS |
| | | RWQQGNVFSCSVMHEALHNH |
| | | YTQKSLSLSPGK |
| 7 | Fc of IgG2 | ERKCCVECPPCPAPPVAGPS |
| | (with hinge | VFLFPPKPKDTLMISRTPEV |
| | region) | TCVVVDVSHEDPEVQFNWYV |
| | | DGVEVHNAKTKPREEQFNST |
| | | FRVVSVLTVVHQDWLNGKEY |
| | | KCKVSNKGLPAPIEKTISKT |
| | | KGQPREPQVYTLPPSREEMT |
| | | KNQVSLTCLVKGFYPSDIAV |
| | | EWESNGQPENNYKTTPPMLD |
| | | SDGSFFLYSKLTVDKSRWQQ |
| | | GNVFSCSVMHEALHNHYTQK |
| | | SLSLSPGK |
| 8 | Fc of IgG3 | ELKTPLGDTTHTCPRCPEPK |
| | (with hinge | SCDTPPPCPRCPEPKSCDTP |
| | region) | PPCPRCPEPKSCDTPPPCPR |
| | | CPAPELLGGPSVFLFPPKPK |
| | | DTLMISRTPEVTCVVVDVSH |
| | | EDPEVQFKWYVDGVEVHNAK |
| | | TKPREEQYNSTFRVVSVLTV |
| | | LHQDWLNGKEYKCKVSNKAL |
| | | PAPIEKTISKTKGQPREPQV |
| | | YTLPPSREEMTKNQVSLTCL |
| | | VKGFYPSDIAVEWESSGQPE |
| | | NNYNTTPPMLDSDGSFFLYS |
| | | KLTVDKSRWQQGNIFSCSVM |
| | | HEALHNRFTQKSLSLSPGK |
| 9 | Fc of IgG4 | ESKYGPPCPSCPAPEFLGGP |
| | (with hinge | SVFLFPPKPKDTLMISRTPE |
| | region) | VTCVVVDVSQEDPEVQFNWY |
| | | VDGVEVHNAKTKPREEQFNS |
| | | TYRVVSVLTVLHQDWLNGKE |
| | | YKCKVSNKGLPSSIEKTISK |
| | | AKGQPREPQVYTLPPSQEEM |
| | | TKNQVSLTCLVKGFYPSDIA |
| | | VEWESNGQPENNYKTTPPVL |
| | | DSDGSFFLYSRLTVDKSRWQ |
| | | EGNVFSCSVMHEALHNHYTQ |
| | | KSLSLSLGK |
| 10 | Fc of IgG1 G1m3 | EPKSCDKTHTCPPCPAPELL |
| | (with hinge | GGPSVFLFPPKPKDTLMISR |
| | region) | TPEVTCVVVDVSHEDPEVKF |
| | | NWYVDGVEVHNAKTKPREEQ |
| | | YNSTYRVVSVLTVLHQDWLN |
| | | GKEYKCKVSNKALPAPIEKT |
| | | ISKAKGQPREPQVYTLPPSR |
| | | EEMTKNQVSLTCLVKGFYPS |
| | | DIAVEWESNGQPENNYKTTP |
| | | PVLDSDGSFFLYSKLTVDKS |
| | | RWQQGNVFSCSVMHEALHNH |
| | | YTQKSLSLSPGK |
| 11 | VH of | EVQLVESGGGLVQPGGSLRL |
| | atezolizumab | SCAASGFTFSDSWIHWVRQA |
| | | PGKGLEWVAWISPYGGSTYY |
| | | ADSVKGRFTISADTSKNTAY |
| | | LQMNSLRAEDTAVYYCARRH |
| | | WPGGFDYWGQGTLVTVSS |
| 12 | VL of | DIQMTQSPSSLSASVGDRVT |
| | atezolizumab | ITCRASQDVSTAVAWYQQKP |
| | | GKAPKLLIYSASFLYSGVPS |
| | | RFSGSGSGTDFTLTISSLQP |
| | | EDFATYYCQQYLYHPATFGQ |
| | | GTKVEIK |
| 13 | VH of | EVQLVESGGGLVQPGGSLRL |
| | durvalumab | SCAASGFTFSRYWMSWVRQA |
| | | PGKGLEWVANIKQDGSEKYY |
| | | VDSVKGRFTISRDNAKNSLY |
| | | LQMNSLRAEDTAVYYCAREG |
| | | GWFGELAFDYWGQGTLVTVSS |
| 14 | VL of | EIVLTQSPGTLSLSPGERAT |
| | durvalumab | LSCRASQRVSSSYLAWYQQK |
| | | PGQAPRLLIYDASSRATGIP |
| | | DRFSGSGSGTDFTLTISRLE |
| | | PEDFAVYYCQQYGSLPWTFG |
| | | QGTKVEIK |
| 15 | VH of avelumab | EVQLLESGGGLVQPGGSLRL |
| | | SCAASGFTFSSYIMMWVRQA |
| | | PGKGLEWVSSIYPSGGITFY |
| | | ADTVKGRFTISRDNSKNTLY |
| | | LQMNSLRAEDTAVYYCARIK |
| | | LGTVTTVDYWGQGTLVTVSS |
| 16 | VL of avelumab | QSALTQPASVSGSPGQSITI |
| | | SCTGTSSDVGGYNYVSWYQQ |
| | | HPGKAPKLMIYDVSNRPSGV |
| | | SNRFSGSKSGNTASLTISGL |
| | | QAEDEADYYCSSYTSSSTRV |
| | | FGTGTKVTVLG |
| 17 | VH of nivolumab | QVQLVESGGGVVQPGRSLRL |
| | | DCKASGITFSNSGMHWVRQA |
| | | PGKGLEWVAVIWYDGSKRYY |
| | | ADSVKGRFTISRDNSKNTLF |
| | | LQMNSLRAEDTAVYYCATND |
| | | DYWGQGTLVTVSS |
| 18 | VL of nivolumab | EIVLTQSPATLSLSPGERAT |
| | | LSCRASQSVSSYLAWYQQKP |
| | | GQAPRLLIYDASNRATGIPA |
| | | RFSGSGSGTDFTLTISSLEP |
| | | EDFAVYYCQQSSNWPRTFGQ |
| | | GTKVEIK |
| 19 | VH of | QVQLVQSGVEVKKPGASVKV |
| | pembrolizumab | SCKASGYTFTNYYMYWVRQA |
| | | PGQGLEWMGGINPSNGGTNF |
| | | NEKFKNRVTLTTDSSTTTAY |
| | | MELKSLQFDDTAVYYCARRD |
| | | YRFDMGFDYWGQGTTVTVSS |
| 20 | VL of | EIVLTQSPATLSLSPGERAT |
| | pembrolizumab | LSCRASKGVSTSGYSYLHWY |
| | | QQKPGQAPRLLIYLASYLES |
| | | GVPARFSGSGSGTDFTLTIS |
| | | SLEPEDFAVYYCQHSRDLPL |
| | | TFGGGTKVEIK |

The invention is illustrated below with the following examples that are in no way limiting.

### Example 1: Production of Anti-PDL1 IgG1 Variants of the Invention in YB2/0 Cells

### A/ Construction of Fc Variants:

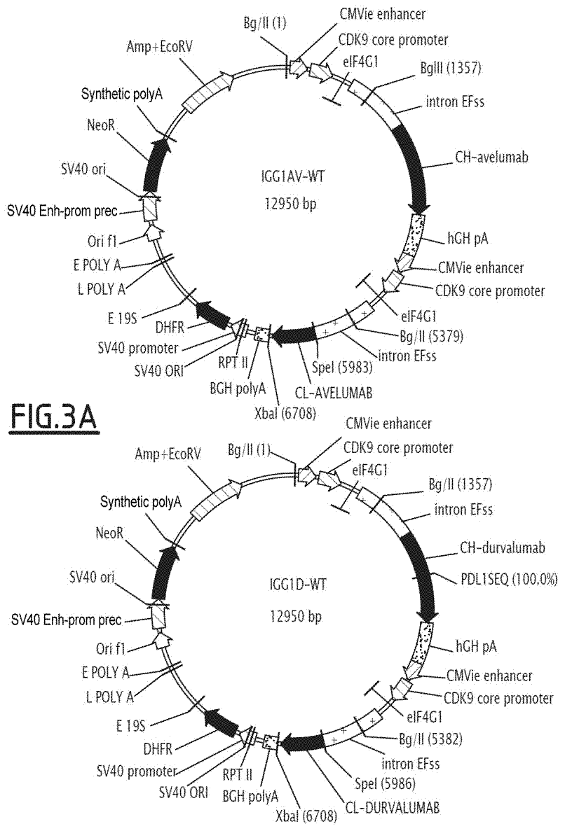

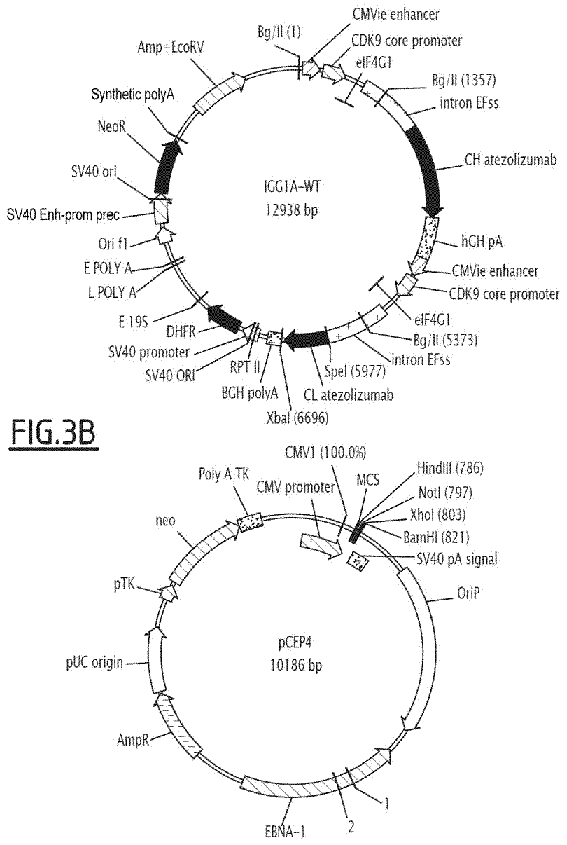

Each mutation of interest in the Fc fragment was independently inserted in an expression vector containing the anti-PDL1 heavy chain (containing the variable part of the anti-PDL1 atezolizumab, durvalumab or avelumab antibody, and the constant part of wild-type Fc region) via overlap extension PCR using two sets of primers adapted to integrate the targeted mutation(s) with the codon(s) encoding the desired amino acid. Advantageously, when the mutations to be inserted are close on the Fc sequence, they are added via one same oligonucleotide. The fragments thus obtained by PCR were associated and the resulting fragment amplified by PCR following standard protocols. The PCR product, containing the whole heavy chain of the anti-PDL1 mutated on the Fc fragment, was purified over 1% agarose gel (w/v), digested with appropriate restriction enzymes and cloned in the eukaryote expression vector (HK-Gen EFSS, see FIG. 3) which also contained the non-modified light chain of the anti-PDL1 antibody under consideration.

### B/ Production of Variants in the Form of Whole Ig in YB2/0:

The Fc variants can be prepared in whole IgG1 format in the YB2/0 cell line (ATCC, CRL-1662) with anti-PDL1 specificity. The production steps via cell culture and purification of the antibodies can be conducted following the techniques described in Example 1 of application WO2001/77181, to produce and select antibodies characterized by a low level of fucosylation at their Asn 297 glycosylation site on the Fc.

The following combinations of mutations are preferably selected to produce the anti-PDL1s in YB2/0:

TABLE 1
Anti-PDL1 mutants of IgG1 selected in particular with
the method described in WO2010/106180

| Variant | Mutations |
|---|---|
| C6A_69 | T307A/N315D/A330V/E382V/N389T/N434Y |
| C6A_78 | T256N/A378V/S383N/N434Y |
| T5A_74 | N315D/A330V/N361D/A378V/N434Y |
| C6A_74 | V259I/N315D/N434Y |
| C6A_60 | P230S/N315D/M428L/N434Y |
| T5A_74A | N315D/N361D/A378V/N434Y |

### Example 2: Production of Anti-PDL1 IgG1 Variants According to the Invention in CHOS or HEK Cells, or in the Milk of Transgenic Animals

The following combinations of mutations are preferably selected:

TABLE 2
Anti-PDL1 mutants of IgG1 selected with the method
described in application WO2016/177984

| Variant | Mutations |
|---|---|
| G3A-103 | K248E, A378V |
| J3A-28 | E333G, A378T, V397M |
| J3B-118A | P396L, N421T, A378V |
| J3B-118 | P396L, N421T |
| A3A-105D | G316D, K326E, A378V |
| A3A-14 | S298N, A378V |
| G3A-95 | I336T, A378V |
| A3A-184A | K334N, P352S, V397M, A378V |
| J3B-23 | N286I, A378V, F423Y |
| K3B-01 | N315D, N361H, P396L |
| G3A-43 | A231V, A378V |
| J3A-06 | A3781, V397M, V412M |
| J3A-16 | N286Y, P352S, A378V |
| O3A-05 | K290E, T366A, A378V |
| Q3A-39 | N286I, P396L, N421T |
| A3A-184 | K334N, P352S, V397M |

Advantageously, the mutants can contain the mutations of the variant T5A-74, C6A-74 or T5A-74A such as given in Table 1, and the mutations of a variant listed in Table 2.

Alternatively, the mutants may contain the mutations of variant T5A-74, C6A-74 or T5A-74A such as given in Table 1, and one of the following mutations: V240M, L242K, L242G, L242F, F243L, E258R, T260A, V262A, K290G, Y296W, S298R or V302R.

### 2-1/ Production of Variants in Whole Ig Form in CHO or HEK

The Fc variants, produced by directed mutagenesis according to the method of Example 1-A, can be prepared in whole IgG1 format in a CHOS cell line (HK-Gen EFSS bicistronic vector) or HEK (using the monocistronic vector pCEP4, a vector containing the light chain and the other similar vector containing the mutated heavy chain, see FIG. 3), with anti-PDL1 specificity. The production steps via cell culture and purification of the antibodies can be conducted following well known techniques and described for example in application WO2016/177984 (Example 2.6 in HEK).

### 2-2/ Production of Variants in Whole Ig Form in Transgenic Animal Milk

The mutated anti-PDL1 whole IgG1s can also be generated by production in the milk of a transgenic animal, e.g. a transgenic goat, and purification via milk extraction. For this purpose, the sequence encoding the heavy chain and the sequence encoding the light chain are prepared in an expression vector under the control of a promoter specific to the mammary glands e.g. a mammalian casein promoter, allowing the production and secretion of the antibody to be directed into the milk of the mammary glands. Said method is particularly described in application EP0741515.

### Example 3A: Methods Allowing Characterization of Binding to FcgRIIIa, of Antigen Binding, and of Binding to FcRn of the Anti-PDL1 IgG1 Variants of the Invention

The binding assays to FcgRIIIa, to the PDL1 ligand and to FcRn were conducted following the methods described in application WO2010/106180. In brief, the binding of the IgG1s to FcgRIIIa, to the PDL1 ligand and to FcRn were measured using a conventional ELISA assay.

Maxisorp immunoplates were coated with PDL1 antigens (pH=7.4) or FcRns (pH=6.0). The solutions of parent anti-PDL1 IgG1 or of each anti-PDL1 IgG1 variant were added to each well to a final concentration of 1.25 μg of IgG/mL for FcRn, or 0.25 μg of IgG/mL for the other receptors, for 1 h at 37° C., then contacted with HRP goat F(ab′)2 anti-human IgG for 1h at 37° C. The bonded IgG1s were detected via TMB visualisation by measuring absorbance at 450 nm.

For binding to FcgRIIIa (CD16a), FcgRIIaH/R (CD32aH/R), FcgRIIb (CD32b) or FcgRI (CD64), Maxisorp or NiNTA immunoplates were coated with FcgRIIIaV/F, FcgRIIaH/R, FcgRIIb or FcgRI, and saturated with 4% PBS-BSA. The solutions of parent anti-PDL1 IgG1 or of each anti-PDL1 Ig G1 variant in a final concentration of 0.5 μg of IgG/mL were contacted with HRP goat F(ab′)2 anti-human IgG at the same concentration for 2 hours at ambient temperature, under gentle agitation. The IgGs aggregated to F(ab′)2 were incubated under gentle agitation for 1 hour at 30° C. on ELISA plates saturated for FcgR. The plates were then visualised with TMB (Pierce) and absorbances read at 450 nm.

The variants of Fc C6A_74 and T5A_74A, comprising the variable part of durvalumab or atezolizumab (i.e. the VH and VL sequences of the durvalumab antibody are the sequences SEQ ID NO: 13 and 14 respectively, and the VH and VL sequences of the atezolizumab antibody are the sequences SEQ ID NO: 11 et 12 respectively), were produced in YB2/0 or in CHO as in Example 1:

| Variant | Mutations |
|---|---|
| C6A_74 | V259I/N315D/N434Y |
| T5A_74A | N315D/N361D/A378V/N434Y |

The reference controls were:
Fc-WT produced in YB2/0,
Fc-WT produced in CHO, but also
aglycosylated Fc comprising the mutation N297A (called «N297A») produced in CHO, and
Fc-FES (i.e. comprising the 3 mutations L234F/L235E/P331S).

The binding thereof to human FcgRIIIa (hCD16aV and hCD16aF) and to human FcRn (hFcRn) was measured.

The results are as follows:

Atezolizumab

| | hCD16aV | hCD16aF | hFcRn | hCD64 | hCD32aR | hCD32b | hCD32aH |
|---|---|---|---|---|---|---|---|
| | (0.25 μg/ml) | (0.25 μg/ml) | (1.25 μg/ml) | (0.25 μg/ml) | (0.25 μg/ml) | (0.25 μg/ml) | (0.25 μg/ml) |
| | ELISA Ratio | ELISA Ratio | ELISA Ratio | ELISA Ratio | ELISA Ratio | ELISA Ratio | ELISA Ratio |
| Ratio/Fc-WT (YB2/0) | | | | | | | |
| Anti-PDL1\|atezolizumab\|Fc-WT (CHOs) | 0.21 | 0.20 | 2.95 | 0.90 | 1.04 | 0.96 | 0.99 |
| Anti-PDL1\|atezolizumab\|C6A-74 (YB2/0) | 0.78 | 0.88 | 13.72 | 1.17 | 1.05 | 0.94 | 0.99 |
| Anti-PDL1\|atezolizumab\|T5A-74A (YB2/0) | 1.03 | 1.57 | 14.60 | 1.51 | 1.04 | 0.97 | 1.00 |
| Anti-PDL1\|atezolizumab\|N297A (CHOs) | 0.02 | 0.22 | 2.10 | 0.01 | 0.11 | 0.09 | 0.10 |
| Ratio/Fc-WT (CHOs) | | | | | | | |
| Anti-PDL1\|atezolizumab\|Fc-WT(YB2/0) | 4.77 | 5.07 | 0.34 | 1.11 | 0.97 | 1.05 | 1.01 |
| Anti-PDL1\|atezolizumab\|C6A-74 (YB2/0) | 3.72 | 4.45 | 4.65 | 1.30 | 1.01 | 0.98 | 1.00 |
| Anti-PDL1\|atezolizumab\|T5A-74A (YB2/0) | 4.90 | 7.99 | 4.95 | 1.68 | 1.00 | 1.02 | 1.01 |
| Anti-PDL1\|atezolizumab\|N297A (CHOs) | 0.10 | 1.10 | 0.71 | 0.01 | 0.11 | 0.10 | 0.10 |
| Ratio/N297A (CHOs) | | | | | | | |
| Anti-PDL1\|atezolizumab\|Fc-WT (CHOs) | 10.1948 | 0.9062 | 1.4045 | 92.5333 | 9.5110 | 10.4443 | 9.9453 |
| Anti-PDL1\|atezolizumab\|Fc-WT(YB2/0) | 48.5989 | 4.5972 | 0.4765 | 103.0556 | 9.1887 | 10.9256 | 9.9969 |
| Anti-PDL1\|atezolizumab\|C6A-74 (YB2/0) | 37.9226 | 4.0364 | 6.5353 | 120.7000 | 9.6156 | 10.2278 | 9.9005 |
| Anti-PDL1\|atezolizumab\|T5A-74A (YB2/0) | 49.9885 | 7.2382 | 6.9571 | 155.1111 | 9.5505 | 10.6143 | 10.0455 |

Durvalumab

| | hCD16aV | hCD16aF | hFcRn | hCD64 | hCD32aR | hCD32b | hCD32aH |
|---|---|---|---|---|---|---|---|
| | (0.25 μg/ml) | (0.25 μg/ml) | (1.25 μg/ml) | (0.25 μg/ml) | (0.25 μg/ml) | (0.25 μg/ml) | (0.25 μg/ml) |
| | ELISA Ratio | ELISA Ratio | ELISA Ratio | ELISA Ratio | ELISA Ratio | ELISA Ratio | ELISA Ratio |
| Ratio/Fc-WT (YB2/0) | | | | | | | |
| Anti-PDL1\|durvalumab\|Fc-WT (CHOs) | 0.23 | 0.17 | 1.83 | 0.81 | 0.99 | 0.96 | 1.08 |
| Anti-PDL1\|durvalumab\|C6A-74 (YB2/0) | 0.90 | 0.86 | 24.11 | 1.15 | 0.98 | 0.93 | 0.90 |
| Anti-PDL1\|durvalumab\|T5A-74A (YB2/0) | 1.15 | 1.70 | 24.81 | 1.63 | 0.99 | 1.01 | 1.72 |
| Anti-PDL1\|durvalumab\|Fc-FES (CHOs) | 0.07 | 0.09 | 1.61 | 0.05 | 0.61 | 0.48 | 0.11 |
| Ratio/Fc-WT (CHOs) | | | | | | | |
| Anti-PDL1\|durvalumab\|Fc-WT (YB2/0) | 4.27 | 5.77 | 0.55 | 1.23 | 1.01 | 1.04 | 0.93 |
| Anti-PDL1\|durvalumab\|C6A-74 (YB2/0) | 3.83 | 4.98 | 13.19 | 1.42 | 0.98 | 0.97 | 0.83 |
| Anti-PDL1\|durvalumab\|T5A-74A (YB2/0) | 4.91 | 9.82 | 13.57 | 2.01 | 1.00 | 1.05 | 1.60 |
| Anti-PDL1\|durvalumab\|Fc-FES (CHOs) | 0.30 | 0.52 | 0.88 | 0.06 | 0.61 | 0.50 | 0.10 |
| Ratio/Fc-FES (CHOs) | | | | | | | |
| Anti-PDL1\|durvalumab\|Fc-WT (CHOs) | 3.3447 | 1.9144 | 1.1391 | 17.6622 | 1.6330 | 2.0023 | 9.8652 |
| Anti-PDL1\|durvalumab\|Fc-WT (YB2/0) | 14.2780 | 11.0367 | 0.6230 | 21.7763 | 1.6425 | 2.0883 | 9.1583 |
| Anti-PDL1\|durvalumab\|C6A-74 (YB2/0) | 12.8024 | 9.5245 | 15.0214 | 25.0716 | 1.6019 | 1.9371 | 8.2253 |
| Anti-PDL1\|durvalumab\|T5A-74A (YB2/0) | 16.4106 | 18.7997 | 15.4601 | 35.4228 | 1.6305 | 2.1121 | 15.7423 |

The results show that the variants C6A_74 and T5A_74A both have equivalent binding to hFcRn, which is strongly increased (more than 12 times), compared with their respective WT parent.

It emerges that all the variants produced in YB2/0 cells have increased binding to hCD16aV and to hCD16aF compared with their respective WT parent produced in CHO cells.

For the variants produced in YB2/0 cells:
C6A_74 has equivalent or slightly reduced binding to hCD16aV and hCD16aF compared with the WT parent, whilst having strongly increased binding to FcRn;
T5A_74A has equivalent binding to hCD16aV, and slightly increased binding to hCD16aF, whilst having strongly increased binding to FcRn.

Similar results were obtained with durvalumab and atezolizumab; they show increased binding to hFcRn and to hCD16aV/F.

### Example 3B: Methods Allowing Characterization of Binding to FcgRIIIa, of Antigen Binding, and of Binding to FcRn of Other Anti-PDL1 IgG1 Variants of the Invention

The same assays as those described in Example 3A were conducted for the following variants, comprising the variable part of durvalumab (i.e. with VH and VL sequences of SEQ ID NO: 13 and 14 respectively) and produced in CHO cells:

| Variant | Mutations |
|---|---|
| C6A_74 | V259I/N315D/N434Y |
| (cited in Ex. 3A) | |
| T5A_74A | N315D/N361D/A378V/N434Y |
| (cited in Ex. 3A) | |
| A3A_184A | K334N/P352S/V397M/A378V |
| C6A_74W | V259I/Y296W/N315D/N434Y |
| A3A_184E | Y296W/K334N/P352S/V397M/A378V |
| T5A_74MA | Y296W/N315D/N361D/A378V/N434Y |
| C6A_74G | V259I/K290G/N315D/N434Y |
| A3A_184AG | K290G/K334N/P352S/V397M/A378V |
| T5A_74AG | K290G/N315D/N361D/A378V/N434Y |
| A3A_184AY | K334N/P352S/V397M/A378V/N434Y |
| A3A_184EY | Y296W/K334N/P352S/V397M/A378V/N434Y |

The parent durvalumab antibody Fc-WT was produced in CHO cells.

The binding thereof to human and murine PD-L1, to human FcgRIIIa (hCD16aV and hCD16aF), to hFcRn, to human CD64 (hCD64), and to hCD32aH/aR/b, was measured.

The results are as follows:

All the anti-PD-L1 antibodies showed strong binding to human PD-L1, and no binding to murine PD-L1.

| | ELISA | ELISA | ELISA |
|---|---|---|---|
| | Ratio | Ratio | Ratio |
| | hCD16aV | hCD16aF | hFcRn |
| | (0.25 | (0.125 | (1.25 |
| | μg/ml) | μg/ml) | μg/ml) |
| Ratio/Fc-WT (CHOs) | mean | mean | mean |
| Anti-PDL1\|durvalunnab\|C6A-74 | 0.83 | 0.87 | 19.07 |
| Anti-PDL1\|durvalunnab\|C6A-74W | 1.69 | 1.28 | 18.55 |
| Anti-PDL1\|durvalunnab\|C6A-74G | 1.32 | 1.10 | 18.78 |
| Anti-PDL1\|durvalunnab\|T5A-74A | 1.39 | 1.17 | 20.05 |
| Anti-PDL1\|durvalunnab\|T5A-74MA | 2.50 | 2.52 | 20.07 |
| Anti-PDL1\|durvalunnab\|T5A-74AG | 2.21 | 2.24 | 19.53 |
| Anti-PDL1\|durvalunnab\|A3A-184EY | 3.02 | 5.20 | 22.70 |
| Anti-PDL1\|durvalunnab\|A3A-184A | 2.52 | 1.99 | 21.55 |
| Anti-PDL1\|durvalunnab\|A3A-184AY | 2.38 | 1.97 | 26.19 |
| Anti-PDL1\|durvalunnab\|A3A-184AG | 1.46 | 0.80 | 17.82 |
| Anti-PDL1\|durvalunnab\|A3A-184E | 2.75 | 4.26 | 19.78 |

| | ELISA | ELISA | ELISA | ELISA |
|---|---|---|---|---|
| | Ratio | Ratio | Ratio | Ratio |
| | hCD64 | hCD32aR | hCD32b | hCD32aH |
| | (0.25 | (0.25 | (0.25 | (0.25 |
| | μg/ml) | μg/ml) | μg/ml) | μg/ml) |
| Ratio/Fc-WT (CHOs) | mean | mean | mean | mean |
| Anti-PDL1\|durvalunnab\|C6A- | 1.17 | 0.92 | 0.92 | 0.87 |
| 74 | | | | |
| Anti-PDL1\|durvalunnab\|C6A- | 1.26 | 0.98 | 0.97 | 0.94 |
| 74W | | | | |
| Anti-PDL1\|durvalunnab\|C6A- | 1.69 | 1.04 | 0.97 | 0.99 |
| 74G | | | | |
| Anti-PDL1\|durvalunnab\|T5A- | 2.09 | 1.01 | 0.99 | 1.03 |
| 74A | | | | |
| Anti-PDL1\|durvalunnab\|T5A- | 2.16 | 1.06 | 1.04 | 1.11 |
| 74MA | | | | |
| Anti-PDL1\|durvalunnab\|T5A- | 2.41 | 1.12 | 0.99 | 1.14 |
| 74AG | | | | |
| Anti-PDL1\|durvalunnab\|A3A- | 5.11 | 1.10 | 1.14 | 1.00 |
| 184EY | | | | |
| Anti-PDL1\|durvalunnab\|A3A- | 3.91 | 1.04 | 1.08 | 1.25 |
| 184A | | | | |
| Anti-PDL1\|durvalunnab\|A3A- | 4.25 | 0.99 | 0.99 | 1.30 |
| 184AY | | | | |
| Anti-PDL1\|durvalunnab\|A3A- | 3.32 | 0.91 | 0.88 | 0.53 |
| 184AG | | | | |
| Anti-PDL1\|durvalunnab\|A3A- | 4.03 | 1.08 | 1.15 | 1.27 |
| 184E | | | | |

All the Durvalumab variants (i.e. the variants of format C6A-74, T5A-74 and A3A-184) have equivalent increased binding to FcRn compared with Fc-WT.

The Durvalumab variants of format A3A-184 show the best affinity for hCD64, followed by the variants of format T5A-74, and finally to a lesser extent by the variants of format C6A-74.

The Durvalumab variants A3A-184E and A3A-184EY show equivalent increased binding to hCD16aF. They represent the best variants. The Durvalumab variants T5A-74MA, T5A-74AG, A3A-184A, A3A-184AY show increased binding to hCD16aF compared with Fc-WT produced in CHO cells.

The Durvalumab variants C6A-74, C6A-74W, C6A-74G and T5A-74A compared with Fc-WT produced in CHO cells show equivalent binding to hCD16aF.

The Durvalumab variants A3A-184E, A3A-184EY, A3A-184A, A3A-184AY and T5A-74MA show equivalent increased binding to hCD16aV compared with Fc-WT.

The Durvalumab variants C6A-74W, C6A-74G, T5A-74A and T5A-74AG show increased binding to hCD16aV compared with Fc-WT.

The Durvalumab variants A3A-184AG and C6A-74 compared with Fc-WT produced in CHO cells have equivalent binding to hCD16aV.

The Durvalumab variants A3A-184A, A3A-184AY, A3A-184E, T5A-74AG and A3A-184EY show slightly increased binding to hCD32aH compared with Fc-WT.

The Durvalumab variants C6A-74, T5A-74MA, C6A-74W, C6A-74G and T5A-74A compared with Fc-WT show equivalent binding to hCD32aH.

All the Durvalumab variants advantageously show maintained binding to hCD32b compared with Fc-WT.

All the Durvalumab variants advantageously show maintained binding to hCD32aR compared with Fc-WT.

To conclude, the variants A3A-184E and A3A-184EY appear to be the best candidates with the strongest binding to hFcRn and hCD16aFN. The variants A3A-184AY and T5A-74MA also appear to be very good candidates with good binding to hFcRn and hCD16aFN. These four variants show strong binding to hCD64 and good binding to hCD32aH. Therefore, these four variants can advantageously be used in the context of treating cancer with an anti-PDL1.

### Example 3C: Method Allowing Characterization of Half-Life

Pharmacokinetic experiments were also conducted in hFcRn mice KO homozygous for an allele of murine FcRn and heterozygous for a transgene of human FcRn (mFcRn′-hFcRnTg), to evaluate the half-life of the atezolizumab variants (i.e. with respective VH and VL sequences SEQ ID NO: 11 and 12) with Fc C6A_74 or T5A_74A according to the invention.

For these pharmacokinetic studies, each animal received a single intravenous IgG injection of 5 mg/kg at the retro-orbital sinus, following a similar protocol to the one previously described (Petkova S B, et al. Enhanced half-life of genetically engineered human IgG1 antibodies in a humanized FcRn mouse model: potential application in humorally mediated autoimmune disease. Int Immunol 2006).

Half-life is generally calculated from plasma concentrations measured during the elimination phase.

Half-life time can therefore be obtained:
by solving the equation:

T½=(Ln 2×Vd)/CL, where:

Vd=distribution volume=Dose/initial plasma concentration

CL=Clearance=Dose/AUC (area under curve)
by graphical analysis by determining on the Y-axis (concentration in μg/ml) the time lapse between concentration C1 and concentration C2. It is essential to plot this curve on semi-logarithmic scale to ensure aligning of the experimental points in this last so-called elimination phase. The investigation time length of this curve must be sufficiently long to allow accurate estimation of half-life.

Once the slope of the elimination phase has been measured (kₑ or elimination rate constant), the half-life can be calculated as follows:

T½=Ln 2/kₑ=0.693/kₑ

The mean residence time, MRT, can also be measured This translates the time the variant is present in the body.

MRT can be obtained as follows:

MRT=AUC/AUMC, where:

AUC=area under the zero moment curve of plasma concentrations as a function of time;

AUMC=area under the first moment curve of plasma concentrations as a function of time.

Blood samples were taken from the retro-orbital sinus at multiple time intervals and the IgGs assayed by ELISA.

The results are the following:

| | T½ | Ratio | Cmax | AUCinf | Vd | CL | MRTinf | Ratio |
|---|---|---|---|---|---|---|---|---|
| Molecule | (h) | T½/WT | (μg/ml) | (h * μg/ml) | (ml/kg) | (ml/h/kg) | (h) | MRTinf/WT |
| Atezolizumab_Fc- | 14.6 | 1.0 | 102.0 | 2007 | 33.4 | 2.51 | 13.5 | 1.0 |
| WT | | | | | | | | |
| Atezoliumab_C6A- | 38.4 | 2.6 | 79.8 | 2564 | 63.4 | 2.06 | 30.5 | 2.3 |
| 74 | | | | | | | | |
| Atezolizumab_T5A- | 41.0 | 2.8 | 92.4 | 3036 | 74.5 | 1.68 | 45.0 | 3.3 |
| 74A | | | | | | | | |

The analysed parameters are defined below:

T½: half-life

Cmax: maximum concentration obtained at a given time, corresponding to the maximum plasma concentration time (Tmax)

AUCinf: Area under time curve/plasma concentration from T0 to infinity

Vd: Distribution volume

Cl: Clearance

MRT: Mean residence time

They show that the atezolizumab variants C6A_74 and T5A_74A have an increase of more than two- to threefold in their half-life compared with the WT parent, respectively. In addition, the atezolizumab variants C6A_74 and T5A_74A show an increase in mean residence time of more than two- to threefold compared with the WT parent, respectively.

### Example 4: Method Allowing Testing of the ADCC Activity of the Anti-PDL1 IgG1 Variants of the Invention

### Example 4A: Methods Using Human Tumour Eukaryote Cells as Target

NK cells are incubated with human tumour eukaryote cells (such as A431 and A549 lines) expressing PDL1, in the presence of different concentrations (0.005 to 5000 ng/ml) of parent anti-PDL1 IgG1 or of each anti-PDL1 IgG1 variant. The level of intracellular lactate dehydrogenase (LDH) released by the lysed target cells is measured.

Human NK cells are purified from the peripheral blood of healthy voluntary donors using the negative depletion technique developed by Miltenyi. The ADCC assay comprises incubation of NK cells with target eukaryote cells expressing the PDL1 antigen, in the presence of different concentrations of anti-PDL1 antibodies. After an incubation time of 16 hours, the cytotoxicity induced by the anti-PDL1 antibodies is measured by quantifying in the cell supernatants the intracellular LDH released by the lysed target cells.

This method can be used efficiently to evaluate the increase in ADCC activity of the antibodies selected in the present application.

### Example 4B: Activation Assay of Jurkat Cells with Stable Surface Expression of the CD16 Receptor (Jurkat-CD16), with Anti-PDL1 Antibodies in the Presence of Target Cells Expressing PDL1

The secretion of IL-2 by Jurkat-CD16 cells in the presence of an antibody composition is known to be correlated with ADCC activity via FcγRIII (CD16) (see WO2004024768, page 3 lines 12-18, page 4 line 29 to page 5 line 2, page 8 lines 25-29, and especially Example 2 pages 14-15 and FIG. 10).

The same antibodies and reference controls as those in Example 3A were characterized for their activation of Jurkat-CD16 cells, by measuring IL-2 secretion.

Target K1 PD-L1 aAPC/CHO cells (25 000 cells/well in 50 μl) were incubated with 50 μl of increasing concentrations of anti-PDL1 antibody (0 to 1250 ng/ml final) in the presence of 50 μl of Jurkat-hCD16aF cells (250 000 cells/well) and 50 μl of PMA (Phorbol-Myristate Acetate) in a final concentration of 10 ng/ml.

After an incubation time of 16 hours at 37° C., the quantity of IL-2 released by Jurkat-CD16 was measured by colorimetry (e.g. RD System DuoSet Kit IL-2: Ref DY202-05). The data are expressed in IL-2 concentration in pg/ml.

The results are the following (expressed in quantity of antibody required to induce 1000 μg of IL-2 (about 50% of maximum level obtained in this assay)):

| | In ng per 1000 pg of |
|---|---|
| | IL-2 |
| Atezolizumab Fc-WT CHO | >1250 |
| Atezolizumab Fc-WT YB2/0 | 2.1 |
| Atezolizumab C6A-74 YB2/0 | 3.4 |
| Atezolizumab T5A-74A YB2/0 | 2.7 |
| Atezolizumab N297A CHO | >1250 |
| Durvalumab FcWT CHO | >1250 |
| Durvalumab Fc-WT YB2/0 | 2.7 |
| Durvalumab C6A-74A YB2/0 | 2 |
| Durvalumab T5A-74A YB2/0 | 2.7 |
| Durvalumab Fc-FES CHO | >1250 |

Results show that the atezolizumab and durvalumab variants C6A_74 and T5A_74A allow IL-2 induction and ADCC activity via CD16 at least equivalent to that of corresponding Fc-WT produced in YB2/0, and much increased compared with that of Fc-WT produced in CHO. Also, the atezolizumab and durvalumab variants produced in YB2/0 have much higher activity than the reference variant atezolizumab N297A CHO and durvalumab Fc-FES CHO respectively.

### Example 5: Antitumor Effect of the Anti-PDL1 IgG1 Variants of the Invention on In Vivo Models

An in vivo tumour model can be used to analyse the effect of the anti-PDL1 variants on animal survival. At Day 0, C57BL/6 nude mice were given intravenous injections of C4198-GFP leukaemia cells. The mice were then separated into 3 groups. At Days 1, 4 and 7, the mice were treated with PBS (vehicle) or with 10 mg/kg of parent anti-PDL1 IgG1 (reference) or with anti-PDL1 variant in a proportion of 200 μL by intraperitoneal injection. The mice were observed twice weekly throughout the entire length of the study (76 days) and on completion the survival rate of the mice was measured. This protocol can allow confirmation in vivo, of the advantage of using an anti-PDL1 antibody that is modified to give improved binding to CD16a and/or improved ADCC activity, compared with a non-modified parent antibody.

### Example 6: Antitumor Effect of the Combination of Anti-PDL1 and Anti-PD1 IgG1 Variants of the Invention on In Vivo Models

### 1-Generation of a Study Model Reproducing a Pathological Situation in Man

A humanized tumour mouse model (HTM)) can be used to analyse the effect on animal survival of combining anti-PDL1 variants (such as described in Examples 1 or 2) and anti-PD1 variants (i.e. variant del294 or N315D/A330V/N361 D/A378V/N434Y or E294deVT307P/N434Y or V259I/N315D/N434Y or V259I/E294Del/N315D/N434Y or N297A, comprising in particular the VH and VL of nivolumab or pembrolizumab such as described in the foregoing description).

This model is characterized by the development of a mature human immune system and the growth of human cancer cells previously co-transplanted with human hematopoietic stem cells.

In brief, NOD-scid IL2Rγnull mice (NSG) can be obtained from Laboratoires Jackson for example and housed in a specialised pathogen-free unit. The new-born mice are irradiated (1 Gy) during their first 48 hours of life and are transplanted 3 hours later via intra-hepatic injection with 2.5×10⁵ human CD34+ cells isolated from umbilical cord blood (UC) in the presence of 3×10⁶ C4198-Luc tumour cells (expressing luciferase for bioluminescence monitoring).

### 2—Method Able to be Used to Test the Activity of the Antibody Combination

As soon as the tumour is visible via bioluminescence (IVIS), the HTM mice are treated with 20 mg/kg of a combination of anti-PD1 and anti-PDL1 variants every 3 days via intravenous route.

Monitoring of treatment efficacy is conducted under bioluminescence; survival of the animals and survival in the absence of tumours are monitored. Blood samples are taken throughout the study to ensure the efficacy of the antibody combination on survival.

### CONCLUSION

The HTM murine model can advantageously be used for in vivo evaluation of the advantage of combining:
the cytotoxic effects of an anti-PDL1 antibody modified to give improved binding to CD16a and/or improved ADCC activity,
the neutralising effects of an anti-PD1 antibody as part of an antitumor treatment.

1-19. (canceled)

20. Antibody directed against a ligand of an immune checkpoint, having a modified Fc fragment compared with that of a parent antibody, and having improved affinity for the FcgRIIIa (CD16a) receptor and/or increased ADCC activity compared with the parent antibody.

21. The antibody according to claim 20, wherein the modified Fc fragment comprises at least one combination of 2 following mutations:
i) a mutation selected from among 307N, 326E, 326T, 334N, 334R, 352L, 378V, 378T, 394P, 396L, 397M, 421T, 434Y and 434S; and
ii) at least one mutation selected from among 226G, 226Y, 227S, 228L, 228R, 230S, 230T, 230L, 231V, 234P, 241L, 243I, 243L, 246R, 246E, 247T, 248E, 253F, 254F, 255W, 259A, 261R, 262A, 263A, 264E, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 330V, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 389K, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434Y, 434S, 435R, 436H, 439R, 440G, 440N, 442F, 442P and 447N,
the numbering being that of the EU Index or Kabat equivalent, and provided that mutation (i) does not take place on the same amino acid as mutation (ii).

22. The antibody according to claim 20, wherein the modified Fc fragment comprises at least one combination of 2 following mutations:
i) a mutation selected from among 307N, 326E, 326T, 334N, 334R, 352L, 378V, 378T, 394P, 396L, 397M and 421T; and
ii) at least one mutation selected from among 226Y, 227S, 230S, 231V, 234P, 243I, 243L, 246R, 246E, 247T, 248E, 253F, 254F, 255W, 259A, 261R, 262A, 263A, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434S, 435R, 436H, 439R, 440G, 440N, 442F, 442P and 447N,
the numbering being that of the EU Index or Kabat equivalent, and provided that mutation (i) does not take place on the same amino acid as mutation (ii).

23. The antibody according to claim 21, wherein the modified Fc fragment comprises at least one combination of 2 following mutations:
i) a mutation selected from among 378V, 378T, 434Y and 434S; and
ii) at least one mutation selected from among 226G, 228L, 228R, 230S, 230T, 230L, 241L, 264E, 307P, 315D, 330V, 362R, 378V, 378T, 389T, 389K, 434Y and 434S,
the numbering being that of the EU Index or Kabat equivalent, and provided that mutation (i) does not take place on the same amino acid as mutation (ii).

24. The antibody according to claim 20, wherein the modified Fc fragment comprises at least one combination of 2 following mutations:
i) a mutation selected from among 378V, 326E, 397M, 334N and 396L; and
ii) at least one mutation selected from among 316D, 397M, 334N, 248E, 231V, 246R, 336T, 421T, 361H, 366A, 439R, 290E, 394P, 307P, 378V, 378T, 286I, 286Y and 298N,
the numbering being that of the EU Index or Kabat equivalent, and provided that mutation (i) does not take place on the same amino acid as mutation (ii).

25. The antibody according to claim 20, wherein the modified Fc fragment comprises:
either a combination of mutations selected from among N315D/A330V/N361D/A378V/N434Y, N315D/N361D/A378V/N434Y, P230S/N315D/M428L/N434Y, T307A/N315D/A330V/E382V/N389T/N434Y, V259I/N315D/N434Y and T256N/A378V/S383N/N434Y;
or the combination of mutations K334N/P352S/V397M/A378V;
or a combination of mutations selected from among N315D/A330V/N361D/A378V/N434Y, V259I/N315D/N434Y, K334N/P352S/V397M/A378V and N315D/N361D/A378V/N434Y, and at least one of the following mutations: K290G, Y296W or N434Y.

26. The antibody according to claim 20, wherein the antibody has improved affinity compared with that of the parent antibody, by a ratio of at least 2.

27. Composition comprising antibodies according to claim 20, said modified Fc fragments having N-glycans on their glycosylation site, wherein said N-glycans have a fucosylation level of less than 65%.

28. The composition according to claim 27, said Fc fragments having N-glycans on their Asn 297 glycosylation site, wherein said N-glycans have a glycan structure of biantennary type with short chains, low sialylation, and non-intercalated terminal N-acetylglucosamines.

29. The composition according to claim 27, said Fc fragments having N-glycans on their Asn 297 glycosylation site, wherein said N-glycans have a content higher than 60% for the forms G0+G1+G0F+G1F, the content of forms G0F+G1F being lower than 50%.

30. The composition according to claim 27, said Fc fragments having N-glycans on their Asn 297 glycosylation site, wherein said N-glycans have a content higher than 60% for the forms G0+G1+G0F+G1F, the fucose content being lower than 65%.

31. The composition according to claim 29, said Fc fragments having N-glycans on their Asn 297 glycosylation site, wherein said N-glycans have a content lower than 40% for the forms G1F+G0F.

32. The antibody according to claim 20, wherein the ligand of the immune checkpoint is selected from among PDL1, OX40L, PDL2, CD80, CD86, galectine-9, MHC II, MHC I, HVEM and adenosine.

33. The antibody according to claim 20, wherein the antibody is an anti-PDL1 comprising a light chain variable sequence (VL) and a heavy chain variable sequence (VH) corresponding to sequences VL and VH of the atezolizumab antibody, durvalumab antibody, or avelumab antibody respectively.

34. Products containing:
a) an antibody according to claim 20, and
b) an antibody directed against an immune checkpoint, having a modified Fc fragment compared with that of a parent antibody, having improved affinity for the FcRn receptor, said immune checkpoint being selected from among PD1, CTLA4, TIM3, LAG3, KIR, BTLA1 and a2AR,
as combination products for simultaneous, separate or time-staggered administration, for use thereof in the prevention or treatment of cancers.

35. The products according to claim 34, wherein the ligand of the immune checkpoint according to a) is PDL1 and wherein the immune checkpoint according to b) is PD1.

36. The antibody according to claim 20, wherein the parent antibodies comprise a parent Fc fragment which is a human Fc fragment.

37. Pharmaceutical composition comprising (i) at least one antibody according to claim 20, and (ii) at least one pharmaceutically acceptable excipient.

38. The antibody according to claim 20, for use thereof in the treatment of cancers.

39. The antibody according to claim 20, wherein the antibody has improved affinity compared with that of the parent antibody, by a ratio higher than 5.


SEQUENCE LISTING (20 sequences)

SEQ ID NO: 1 - protein, 222 aa, Artificial Sequence
  Fc of IgG1 G1m1,17
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 221 aa, Artificial Sequence
  Fc of IgG2
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 222 aa, Artificial Sequence
  Fc of IgG3
CPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 222 aa, Artificial Sequence
  Fc of IgG4
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 222 aa, Artificial Sequence
  Fc of IgG1 G1m3
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Fc of IgG1 G1m1,17
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, Artificial Sequence
  Fc of IgG2
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 279 aa, Artificial Sequence
  Fc of IgG3
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 229 aa, Artificial Sequence
  Fc of IgG4
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 232 aa, Artificial Sequence
  Fc of IgG1 G1m3
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 118 aa, Artificial Sequence [sequence omitted]

SEQ ID NO: 12 - protein, 107 aa, Artificial Sequence [sequence omitted]

SEQ ID NO: 13 - protein, 121 aa, Artificial Sequence
  VH of durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 14 - protein, 108 aa, Artificial Sequence
  VL of durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 15 - protein, 120 aa, Artificial Sequence
  VH of avelumab
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 16 - protein, 111 aa, Artificial Sequence
  VL of avelumab
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLG

SEQ ID NO: 17 - protein, 113 aa, Artificial Sequence [sequence omitted]

SEQ ID NO: 18 - protein, 107 aa, Artificial Sequence [sequence omitted]

SEQ ID NO: 19 - protein, 120 aa, Artificial Sequence [sequence omitted]

SEQ ID NO: 20 - protein, 111 aa, Artificial Sequence [sequence omitted]